(19) United States
(12) Patent Application Publication
(10) Pub. No.: US 2026/0256869 A1
(43) Pub. Date: Sep. 3, 2026

(54) ANTAGONISTS OF BACTERIAL PATHOGEN TYPE III SECRETION SYSTEM NEEDLE ASSEMBLY

(71) Applicant: Georgia Tech Research Corporation, Atlanta, GA (US)

(72) Inventor: Andrew McShan, Atlanta, GA (US)

(21) Appl. No.: 19/553,598

(22) Filed: Mar. 2, 2026

Related U.S. Application Data
(60) Provisional application No. 63/764,752, filed on Feb. 28, 2025.

(51) Int. Cl. A61K 38/00; A61K 38/17; C12N 15/10

(52) U.S. Cl. CPC A61K 38/00; A61K 38/1709; C12N 15/1037

(57) ABSTRACT

Peptides for inhibiting type 3 secretion systems (T3SS) in bacteria and methods of using the same.

### CROSS-REFERENCE TO RELATED APPLICATIONS

This application claims benefit of U.S. Provisional Application No. 63/764,752, filed Feb. 28, 2025, incorporated herein by reference in its entirety.

### REFERENCE TO SEQUENCE LISTING

The sequence listing submitted on Mar. 2, 2026, as an .XML file entitled “10034-394US1 ST26.xml” created on Feb. 9, 2026 and having a file size of 56,328 bytes is hereby incorporated by reference pursuant to 37 C.F.R. § 1.52 (e) (5).

### BACKGROUND

The type III secretion system (T3SS) is a multicomponent protein apparatus utilized by Gram-negative bacteria pathogens to directly secrete effector proteins into host cells through pores formed in the host cell membrane. Homologous T3SS systems are found in many Gram-negative bacterial pathogens including causative agents of typhoid fever (Salmonella serotype Typhi), bacillary dysentery (Shigella dysenteriae), whooping cough (Bordetella pertussis), melioidosis (Burkholderia pseudomallei), pneumonia (Pseudomonas aeruginosa), sexually transmitted infections (Chlamydia trachomatis), and bubonic plague (Yersinia pestis). Infection by Gram-negative bacterial pathogens harboring T3SS systems results in high mortality in humans (>1 million deaths) and significant economic burden (>$10 billion) worldwide annually. Conventional strategies for treatment in a clinical setting involve a strict regimen of antibiotics. However, overuse of antibiotics has resulted in the rapid evolution of multidrug resistance, resulting in an arms race between microbes, researchers, and clinicians. Thus, changes are urgently needed in thinking about targeting bacterial pathogens.

### SUMMARY

In one aspect, disclosed herein are peptides comprising at least 80% sequence identity to any one of SEQ ID NOs: 1-28 or a variant thereof having at least 90% sequence identity to any one of SEQ ID NOs: 1-28.

Also disclosed herein, in one aspect, are peptides (including, but not limited to monomeric peptides and peptides comprising a coiled coil and/or three alpha helices) comprising an N-terminal alpha helix positioned adjacent an N-terminus of the peptide; a C-terminal alpha helix positioned adjacent a C-terminus of the peptide; and a middle alpha helix positioned after the N-terminal alpha helix and before the C-terminal alpha helix; wherein the N-terminal alpha helix is from about 20 to about 25 amino acids in length; wherein the C-terminal alpha helix is from about 20 to about 25 amino acids in length; and wherein the middle alpha helix is from about 20 to about 25 amino acids in length. In one aspect, the peptide has a dissociation constant (K_{D}) with PrgI of from about 1 μM to about 200 μM.

In one aspect, disclosed herein are peptides of any preceding aspect, wherein the N-terminal alpha helix is joined to the middle alpha helix by a first linker, and wherein the middle alpha helix is joined to the C-terminal alpha helix by a second linker.

Also disclosed herein are peptides comprising three alpha helices, wherein the peptide (including, but not limited to monomeric peptides and peptides comprising a coiled coil and/or three alpha helices) interacts with one, two, three, four, five, six, seven, eight, nine, ten or more of V12, K15, G19, Q48, S49, E53, L56, A60, I71, A74, or 175 of PrgI chain D (SEQ ID NO: 29). In some aspects, each amino acid in the peptide which interacts with PrgI chain D is independently from about 5 Å to about 12 Å away from PrgI chain D. In one aspect, the peptide has a dissociation constant (K_{D}) with PrgI of from about 1 μM to about 200 μM.

In one aspect, disclosed herein are peptides of any preceding aspect, wherein the peptide can be expressed by a bacterium (such as, for example E. coli) or a yeast.

Also disclosed herein are peptides of any preceding aspect, wherein the peptide (including, but not limited to monomeric peptides and peptides comprising a coiled coil) comprises: an N-terminal alpha helix positioned adjacent an N-terminus of the peptide; a C-terminal alpha helix positioned adjacent a C-terminus of the peptide; and a middle alpha helix positioned after the N-terminal alpha helix and before the C-terminal alpha helix.

In one aspect, disclosed herein are peptides of any preceding aspect, wherein the N-terminal alpha helix, the C-terminal alpha helix, and/or the middle alpha helix is from about 20 to about 25 amino acids in length. In one aspect, the N-terminal alpha helix is joined to the middle alpha helix by a first linker, and wherein the middle alpha helix is joined to the C-terminal alpha helix by a second linker.

Also disclosed herein are peptides of any preceding aspect, wherein each amino acid in the peptide which interacts with PrgI chain D is independently from about 5 Å to about 12 Å away from PrgI chain D.

In one aspect, disclosed herein are peptides of any preceding aspect, wherein the peptide can be expressed by a bacterium (such as, for example E. coli) or a yeast.

In one aspect, disclosed herein are vectors encoding the peptide of any preceding aspect.

Also disclosed herein are cells (such as a bacterium (including, but not limited to E. coli or a yeast) comprising the vector of any preceding aspect.

In one aspect, disclosed herein are methods of inhibiting type 3 secretion system (T3SS) in a bacterium, the method comprising administering to the bacterium the peptide of any preceding aspect. In some aspects the method occurs in vitro, in vivo, or ex vivo.

Also disclosed herein are methods of treating, inhibiting, reducing, decreasing, and/or ameliorating a bacterial infection in a subject (including a mammalian subject such as, for example, a human) in need thereof, the method comprising administering to the subject the peptide of any preceding aspect.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIGS. 1A-1B depict approaches for de novo design of proteins binders of Salmonella T3SS needle protein PrgI. FIG. 1A shows the cryo-EM structure of the PrgI (needle) complex forming extracellular portion of the Salmonella T3SS apparatus (PDB ID 7RYE). Three interacting PrgI subunits are colored orange and blue highlighting two opposing interfaces responsible for stabilizing the needle complex. Other PrgI units are colored gray for clarity. Residues involved in hydrogen bonding/salt bridges between the subunits are noted with sticks. A surface view of the interaction is also presented. FIG. 1B shows a summary of two approaches for de novo design of PrgI binding proteins. Left: in approach I (“rosempn” designs), the orange PrgI subunit is kept fixed as the target while the α-helix of an adjacent blue PrgI subunit is used as a scaffold/motif to design α-helical bundles. Here, the sequence for the scaffold helix is eventually redesigned. The design process involves RosettaDesign (backbone), ProteinMPNN (sequence), and AlphaFold2 (fold validation). Right: in approach II (“rdmpn” designs), hotspots are defined on the target orange PrgI subunit to diffuse a variety of backbones for the binder. The design process involves RFDiffusion (backbone), ProteinMPNN (sequence), and AlphaFold2 (fold validation).

FIGS. 2A-2D depict in silico characterization of de novo designed “rdmpn” and “rosempn” proteins. FIG. 2A shows PDBe PISA (Proteins, Interfaces, Structures and Assemblies) analysis of models of the PrgI*/design interfaces for 28 de novo designed proteins experimentally tested as candidate PrgI* binders. FIG. 2B shows Rosetta InterfaceAnalyzer analysis of models of the PrgI*/design interfaces the 28 de novo designed proteins experimentally tested as candidate PrgI* binders. FIGS. 2C-2D show representative results from all-atom molecular dynamics simulations performed in explicit solvent for PrgI*/design complexes with backbone root mean square deviation (RMSD) analysis (FIG. 2C) and C_{α}-C_{α} distance measurements spanning the interface (FIG. 2D). Backbone structures of rosempn (purple) and rdmpn (pink) designs with their intended binding modes to the Salmonella needle protein PrgI (orange) are shown.

FIG. 3 depicts molecular models of de novo designed “rdmpn” and “rosempn” proteins with PrgI. Backbone structures of rosempn (purple) and rdmpn (pink) designs with their intended binding modes to the Salmonella needle protein PrgI (orange).

FIGS. 4A-4C depict SDS-PAGE of recombinantly expressed and purified PrgI* and de novo designed “rdmpn” proteins. FIG. 4A shows a representative example of purification of the His₆-GB1-PrgI* construct. The His₆-GB1 tag was removed using TEV protease. PrgI* was purified by Nickle-NTA affinity following cleavage. FIG. 4B shows examples of highly expressed “rdmpn” designs. FIG. 4C shows examples of moderately expressed and non-expressed “rdmpn” designs. In FIGS. 4B-4C, “before” and “after” denote pre- and post-purification via Ni-NTA affinity purification.

FIGS. 5A-5D depict de novo designed PrgI binders are folded and stable α-helical proteins. FIG. 5A shows examples models of PrgI (orange) in complex with rdmpn (pink) and rosempn (purple) designs. FIG. 5B shows representative size exclusion chromatograms performed using Superdex 200 Increase 10/300 GL at 0.5 mL/min for each design. FIG. 5C shows representative far-UV CD spectra of each design (0.3 mg/mL) recorded in the absence of PrgI*. The CD derived θ₂₂₂/θ₂₀₈ ratio for each design is noted. FIG. 5D shows CD based thermal melt curves recorded at 222 nm from 25° C. to 95° C. for each design (0.3 mg/mL) recorded in the absence of PrgI*.

FIG. 6 depicts size exclusion chromatography of de novo designed “rdmpn” and “rosempn” proteins. Size exclusion chromatograms performed using Superdex 200 Increase 10/300 GL column at 0.5 mL/min for each design. Design proteins, each with their own unique molecular weights, are expected to elute between 28 and 38 minutes as monomeric species.

FIG. 7 depicts circular dichroism spectra of PrgI* and de novo designed “rdmpn” and “rosempn” proteins. Far-UV CD spectra of each protein (0.3 to 0.5 mg/mL) is shown. The CD derived θ₂₂₂/θ₂₀₈ ratio for each protein is noted. θ₂₂₂/θ₂₀₈ ratio greater than 1.0 typically indicates an α-helical coiled coil, while a θ₂₂₂/θ₂₀₈ ratio <0.9 or less indicates the presence of a smaller bundle (or an α-helix in isolation).

FIG. 8 depicts thermal stability of de novo designed “rdmpn” and “rosempn” proteins. CD derived thermal melt curves recorded at 222 nm from 25° C. to 95° C. for each protein (0.3 to 0.5 mg/mL) are shown. Melting temperature (Tₘ) values were determined from fitting with a Boltzmann Sigmoidal equation. N/D=melting temperature could not be determined, but likely indicates a Tₘ value greater than 80° C.

FIGS. 9A-9C depict experimental validation of PrgI* binding to rdmpn designs with varying affinities. FIG. 9A shows representative SPR sensorgrams of immobilized PrgI*-biotin titrated with increasing concentrations of a high affinity binder (rdmpn.r1.4_1), a moderate affinity binder (rdmpn.r1.5_3) and a non-binder (rdmpn.9). RU=response units. FIG. 9B shows fits from one-site binding model analysis of the SPR data. FIG. 9C shows a summary of SPR derived dissociation constants (K_{D}, μM) comparing binding affinities for high affinity, moderate affinity, and weak affinity binders. Non-binders are excluded. Representative backbone models of high affinity designs are shown. Data are mean±standard deviation for n=3 technical replicates. The K_{D} values are also summarized in TABLE 1.

FIGS. 10A-10C show solution NMR mapping of de novo designed binders with PrgI*. Top: representative positive binder (rdmpn.r2.6_2); Bottom: representative weak or non-binder (rdmpn.r2.8_2). FIG. 10A shows 2D ¹H-¹⁵N amide HSQC spectra of 200 μM ¹⁵N-labeled PrgI* in the absence (orange) and presence of 2-fold molar excess design protein (blue). FIG. 10B shows chemical shift perturbations (CSP, ppm) of ¹⁵N-labeled PrgI* in the presence of 2-fold molar excess design protein relative to unbound PrgI* plotted as a function of PrgI* residue number. The black dotted line represents the average CSP plus one standard deviation for titrations with rdmpn.r2.6_2 (the “best” binder).

FIGS. 11A-11B depict designs that inhibit in vitro polymerization of PrgI* needles. FIG. 11A, left shows a cartoon schematic of the assembly experiment. PrgI* (0.8 mg/ml) was incubated at 25° C. for a week to allow in vitro polymerization in the absence or presence of 3-fold molar excess of design proteins. FIG. 11A, right shows TEM images of PrgI* following the assembly experiment in the absence and presence of different design proteins. FIG. 11B, left shows a cartoon schematic of the disassembly experiment. PrgI* (0.8 mg/ml) was incubated at 25° C. for a week to allow in vitro polymerization in the absence of designs. After one week, 3-fold molar excess of design proteins were added. FIG. 11B, right shows TEM images of PrgI* following the disassembly experiment in the absence and presence of different design proteins. Yellow arrows denote no visible change on formation of in vitro polymerized PrgI* needles, while green arrows denote an observable change. TEM, scale bar=200 nm.

FIGS. 12A-12C depict putative mechanisms of “rdmpn” design inhibition of PrgI needle assembly. FIGS. 12A-12B show hydrogen bond and salt bridge polar contacts (cyan dotted lines) between PrgI (orange) and high affinity binders (pink) rdmpn.r1.4_1, and rdmpn.r2.1_1 as determined by PDBe PISA. FIGS. 12C-12D show PrgI/design models superimposed onto the target PrgI chain (orange) of the cryo-EM structure of the PrgI needle (PDB 7RYE). The design sterically hinders interaction of the target PrgI chain with nearby PrgI subunits (light blue).

FIGS. 13A-13B show sequence and structural conservation of Inv-Mxi-Spa family T3SS needle proteins. FIG. 13A shows Clustal Omega (v1.2.4) sequence alignment of T3SS needle sequences: PrgI from Salmonella typhimurium (UniProt #P41784), BsaL from Burkholderia pseudomallei (UniProt #Q63K18), MxiH from Shigella flexneri (UniProt #P0A223), CprI from Chromobacterium haemolyticum (UniProt #A0A2T5NVZ6), YsaG from Sodalis glossinidius (UniProt #Q6R8E1), EprI from Escherichia coli (UniProt #A0A0H3JK13), ErgI from Erwinia amylovora (UniProt #D4HX33), XrgI from Xanthomonas oryzae (UniProt #A0A3T0G4C3), EsaG from Edwardsiella ictaluri (UniProt #Q1ACA2), and PsaG from Pantoea ananas (UniProt #A0A0D5A0K5). The secondary structure of PrgI from PDB ID 2JOW is shown. The alignment was processed in ESPript v3. FIG. 13B shows experimental structures (PrgI—PDB ID 2JOW; BsaL—PDB ID 2G0U; MxiH—PDB ID 2CA5) or AlphaFold2 models of different T3SS needle proteins.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of a disease disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

The term “virulence” refers to the degree or capacity of a microorganism, pathogen, or biological agent to cause disease or damage in a host organism. “Virulence” encompasses the measurable traits, factors, or mechanisms that contribute to the severity of the infection, including but not limited to the organism's ability to invade host tissues, evade host immune responses, replicate within the host environment, or produce toxins or other pathogenic determinants. As used herein, “virulence” may be quantitatively or qualitatively assessed using any suitable biological, molecular, or phenotypic indicators known in the art or described in the present application. Virulence is commonly quantified by infectious dose 50 (ID50), the number of organisms required to infect 50% of a host population. “Virulence” is distinct from “pathogenicity”, which describes if a microorganism is capable of causing disease.

The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

Reference also is made herein to peptides, polypeptides, proteins and compositions comprising peptides, polypeptides, and proteins. As used herein, a polypeptide and/or protein is defined as a polymer of amino acids, typically of length ≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

A “functional fragment” as referred to herein comprises a portion of a polypeptide which retains its functional ability. In this case, the functional fragment would retain the ability to perform as a telomerase.

As disclosed herein, exemplary peptides, polypeptides, proteins may comprise, consist essentially of, or consist of any reference amino acid sequence disclosed herein, or variants of the peptides, polypeptides, and proteins may comprise, consist essentially of, or consist of an amino acid sequence having at least about 80%, 90%, 95%, 96%, 97%, 98%, or 99% sequence identity to any amino acid sequence disclosed herein. Variant peptides, polypeptides, and proteins may include peptides, polypeptides, and proteins having one or more amino acid substitutions, deletions, additions and/or amino acid insertions relative to a reference peptide, polypeptide, or protein. Also disclosed are nucleic acid molecules that encode the disclosed peptides, polypeptides, and proteins (e.g., polynucleotides that encode any of the peptides, polypeptides, and proteins disclosed herein and variants thereof).

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides. An insertion or addition may refer to 1, 2, 3, 4, 5, 10, 20, 30, 40, 50, 60, 70, 80, 90, 100, 150, or 200 amino acid residues or nucleotides.

Fusion proteins and fusion polynucleotides also are contemplated herein. A “fusion protein” refers to a protein formed by the fusion of at least one peptide, polypeptide, protein or variant thereof as disclosed herein to at least one molecule of a heterologous peptide, polypeptide, protein or variant thereof. The heterologous protein(s) may be fused at the N-terminus, the C-terminus, or both termini. A fusion protein comprises at least a fragment or variant of the heterologous protein(s) that are fused with one another, preferably by genetic fusion (i.e., the fusion protein is generated by translation of a nucleic acid in which a polynucleotide encoding all or a portion of a first heterologous protein is joined in-frame with a polynucleotide encoding all or a portion of a second heterologous protein). The heterologous protein(s), once part of the fusion protein, may each be referred to herein as a “portion”, “region” or “moiety” of the fusion protein.

A fusion polynucleotide refers to the fusion of the nucleotide sequence of a first polynucleotide to the nucleotide sequence of a second heterologous polynucleotide (e.g., the 3′ end of a first polynucleotide to a 5′ end of the second polynucleotide). Where the first and second polynucleotides encode proteins, the fusion may be such that the encoded proteins are in-frame and results in a fusion protein. The first and second polynucleotide may be fused such that the first and second polynucleotide are operably linked (e.g., as a promoter and a gene expressed by the promoter as discussed below).

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2nd ed., vol. 1 3, Cold Spring Harbor Press, Plainview N.Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

“Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

### Peptides and Methods of Use Thereof

In one aspect, provided is a peptide including at least 80% sequence identity (e.g., at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, 100% sequence identity) to any one of SEQ ID NOs: 1-28. In some aspects, the peptide includes any one of SEQ ID NOs: 1-28. In some aspects, the peptide consists of any one of SEQ ID NOs: 1-28.

In another aspect, provided is a peptide including: an N-terminal alpha helix positioned adjacent an N-terminus of the peptide; a C-terminal alpha helix positioned adjacent a C-terminus of the peptide; and a middle alpha helix positioned after the N-terminal alpha helix and before the C-terminal alpha helix. In some aspects, the N-terminal alpha helix can be from about 20 to about 25 amino acids in length, the C-terminal alpha helix is from about 20 to about 25 amino acids in length, and the middle alpha helix can be from about 20 to about 25 amino acids in length. In some aspects, the peptide can interact with one or more (e.g., two or more, three or more, four or more, five or more, six or more, seven or more, eight or more, nine or more, ten or more) of V12, K15, G19, Q48, S49, E53, L56, A60, I71, A74, or 175 of PrgI chain D (SEQ ID NO: 29). In some aspects, the peptide can interact with each of V12, K15, G19, Q48, S49, E53, L56, A60, I71, A74, and I75 of PrgI chain D (SEQ ID NO: 29).

In yet another aspect, provided is a peptide including three alpha helices, wherein the peptide interacts with one or more (e.g., two or more, three or more, four or more, five or more, six or more, seven or more, eight or more, nine or more, ten or more) of V12, K15, G19, Q48, S49, E53, L56, A60, I71, A74, or 175 of PrgI chain D (SEQ ID NO: 29). In some aspects, the peptide can interact with each of V12, K15, G19, Q48, S49, E53, L56, A60, I71, A74, and I75 of PrgI chain D (SEQ ID NO: 29). In some aspects, the peptide can include: an N-terminal alpha helix positioned adjacent an N-terminus of the peptide; a C-terminal alpha helix positioned adjacent a C-terminus of the peptide; and a middle alpha helix positioned after the N-terminal alpha helix and before the C-terminal alpha helix.

In some aspects, the N-terminal alpha helix of any of the disclosed peptides can be about 20 amino acids or more in length (e.g., about 21 amino acids or more, about 22 amino acids or more, about 23 amino acids or more, about 24 amino acids or more, about 25 amino acids or more). In some aspects, the N-terminal alpha helix of any of the disclosed peptides can be about 25 amino acids or less in length (e.g., about 24 amino acids or less, about 23 amino acids or less, about 22 amino acids or less, about 21 amino acids or less, about 20 amino acids or less). It is considered that the N-terminal alpha helix of any of the disclosed peptides can have a length ranging from any of the minimum values described above to any of the maximum values described above. For example, in some aspects, the N-terminal alpha helix of any of the disclosed peptides can be from about 20 to about 25 amino acids in length (e.g., from about 21 to about 24 amino acids in length, from about 22 to about 23 amino acids in length, from about 20 to about 23 amino acids in length, from about 21 to about 22 amino acids in length, from about 22 to about 25 amino acids in length, from about 23 to about 24 amino acids in length).

In some aspects, the C-terminal alpha helix of any of the disclosed peptides can be about 20 amino acids or more in length (e.g., about 21 amino acids or more, about 22 amino acids or more, about 23 amino acids or more, about 24 amino acids or more, about 25 amino acids or more). In some aspects, the C-terminal alpha helix of any of the disclosed peptides can be about 25 amino acids or less in length (e.g., about 24 amino acids or less, about 23 amino acids or less, about 22 amino acids or less, about 21 amino acids or less, about 20 amino acids or less). It is considered that the C-terminal alpha helix of any of the disclosed peptides can have a length ranging from any of the minimum values described above to any of the maximum values described above. For example, in some aspects, the C-terminal alpha helix of any of the disclosed peptides can be from about 20 to about 25 amino acids in length (e.g., from about 21 to about 24 amino acids in length, from about 22 to about 23 amino acids in length, from about 20 to about 23 amino acids in length, from about 21 to about 22 amino acids in length, from about 22 to about 25 amino acids in length, from about 23 to about 24 amino acids in length).

In some aspects, the middle alpha helix of any of the disclosed peptides can be about 20 amino acids or more in length (e.g., about 21 amino acids or more, about 22 amino acids or more, about 23 amino acids or more, about 24 amino acids or more, about 25 amino acids or more). In some aspects, the middle alpha helix of any of the disclosed peptides can be about 25 amino acids or less in length (e.g., about 24 amino acids or less, about 23 amino acids or less, about 22 amino acids or less, about 21 amino acids or less, about 20 amino acids or less). It is considered that the middle alpha helix of any of the disclosed peptides can have a length ranging from any of the minimum values described above to any of the maximum values described above. For example, in some aspects, the middle alpha helix of any of the disclosed peptides can be from about 20 to about 25 amino acids in length (e.g., from about 21 to about 24 amino acids in length, from about 22 to about 23 amino acids in length, from about 20 to about 23 amino acids in length, from about 21 to about 22 amino acids in length, from about 22 to about 25 amino acids in length, from about 23 to about 24 amino acids in length).

In some aspects, the N-terminal alpha helix of any of the disclosed peptides can be joined to the middle alpha helix of any of the disclosed peptides by a first linker, and the middle alpha helix of any of the disclosed peptides can be joined to the C-terminal alpha helix of any of the disclosed peptides by a second linker. In some aspects, any of the disclosed peptides can be monomeric.

In some aspects, any of the disclosed peptides can be a coiled coil. The term “coiled coil” refers to a peptide/protein sequence usually with a contiguous pattern of hydrophobic residues spaced 3 and 4 residues apart, which assembles (folds) to form a multi-meric bundle of helices. Coiled coil structures of polypeptides can be characterized by heptad repeats represented by the consensus sequence (abcdefg)ₙ, with generally hydrophobic residues in position a and d, and generally polar residues at the remaining positions.

In some aspects, any of the disclosed peptides consists of three alpha helices.

In some aspects, any of the disclosed peptides can have a dissociation constant (K_{D}) with PrgI of about 1 μM or more (e.g., about 2 μM or more, about 3 μM or more, about 4 μM or more, about 5 μM or more, about 10 UM or more, about 15 μM or more, about 20 μM or more, about 25 μM or more, about 30 μM or more, about 35 μM or more, about 40 μM or more, about 45 μM or more, about 50 μM or more, about 60 μM or more, about 70 UM or more, about 80 μM or more, about 90 μM or more, about 100 μM or more, about 110 μM or more, about 120 μM or more, about 130 μM or more, about 140 μM or more, about 150 μM or more, about 160 μM or more, about 170 μM or more, about 180 μM or more, about 190 μM or more, about 200 μM or more). In some aspects, any of the disclosed peptides can have a dissociation constant (K_{D}) with PrgI of about 200 μM or less (e.g., about 190 μM or less, about 180 μM or less, about 170 μM or less, about 160 μM or less, about 150 μM or less, about 140 μM or less, about 130 μM or less, about 120 μM or less, about 110 μM or less, about 100 μM or less, about 90 μM or less, about 80 μM or less, about 70 μM or less, about 60 μM or less, about 50 μM or less, about 45 μM or less, about 40 μM or less, about 35 μM or less, about 30 μM or less, about 25 μM or less, about 20 μM or less, about 15 μM or less, about 10 μM or less, about 5 μM or less, about 4 μM or less, about 3 μM or less, about 2 μM or less, about 1 μM or less).

It is considered that any of the disclosed peptides can have a dissociation constant (K_{D}) with PrgI ranging from any of the minimum values described above to any of the maximum values described above. For example, in some aspects, any of the disclosed peptides can have a dissociation constant (K_{D}) with PrgI of from about 1 μM to about 200 μM (e.g., from about 2 μM to about 190 μM, from about 3 μM to about 180 μM, from about 4 μM to about 170 μM, from about 5 μM to about 160 μM, from about 10 μM to about 150 μM, from about 15 M to about 140 μM, from about 20 μM to about 130 μM, from about 25 μM to about 120 M, from about 30 μM to about 110 μM, from about 35 μM to about 100 μM, from about 40 μM to about 90 μM, from about 45 μM to about 80 μM, from about 50 μM to about 70 μM, from about 1 μM to about 60 μM, from about 2 μM to about 50 μM, from about 3 μM to about 45 μM, from about 4 μM to about 40 μM, from about 5 μM to about 35 μM, from about 10 μM to about 30 μM, from about 15 μM to about 25 μM, from about 60 μM to about 200 μM, from about 70 μM to about 190 μM, from about 80 μM to about 180 μM, from about 90 μM to about 170 μM, from about 100 μM to about 160 μM, from about 110 μM to about 150 μM, from about 120 μM to about 140 μM).

In some aspects, each amino acid in any of the disclosed peptides which interacts with PrgI chain D can independently be about 5 Å or more away from PrgI chain D (e.g., about 5.5 Å or more, about 6 Å or more, about 6.5 Å or more, about 7 Å or more, about 7.5 Å or more, about 8 Å or more, about 8.5 Å or more, about 9 Å or more, about 9.5 Å or more, about 10 Å or more, about 10.5 Å or more, about 11 Å or more, about 11.5 Å or more, about 12 Å or more). In some aspects, each amino acid in any of the disclosed peptides which interacts with PrgI chain D can independently be about 12 Å or less away from PrgI chain D (e.g., about 11.5 Å or less, about 11 Å or less, about 10.5 Å or less, about 10 Å or less, about 9.5 Å or less, about 9 Å or less, about 8.5 Å or less, about 8 Å or less, about 7.5 Å or less, about 7 Å or less, about 6.5 Å or less, about 6 Å or less, about 5.5 Å or less, about 5 Å or less).

It is considered that each amino acid in any of the disclosed peptides which interacts with PrgI chain D can independently be a distance away from PrgI chain D ranging from any of the minimum values described above to any of the maximum values described above. For example, in some aspects, each amino acid in any of the disclosed peptides which interacts with PrgI chain D can independently be from about 5 Å to about 12 Å away from PrgI chain D (e.g., from about 5.5 Å to about 11.5 Å, from about 6 Å to about 11 Å, from about 6.5 Å to about 10.5 Å, from about 7 Å to about 10 Å, from about 7.5 Å to about 9.5 Å, from about 8 Å to about 9 Å, from about 5 Å to about 9 Å, from about 5.5 Å to about 8.5 Å, from about 6 Å to about 8 Å, from about 6.5 Å to about 7.5 Å, from about 8 Å to about 12 Å, from about 8.5 Å to about 11.5 Å, from about 9 Å to about 11 Å, from about 9.5 Å to about 10.5 Å).

In some aspects, any of the disclosed peptides can be expressed by a bacterium or a yeast. In some aspects, any of the disclosed peptides can be expressed by E. coli.

In yet another aspect, provided is a vector encoding any of the disclosed peptides. In yet still another aspect, provided is a cell including any of the disclosed vectors. In some aspects, the cell can be a bacterium or a yeast. In some aspects, the cell can be E. coli.

In yet another aspect, provided is a method of inhibiting type 3 secretion system (T3SS) in a bacterium, the method including administering to the bacterium any of the disclosed peptides or vectors. In some examples, the method can occur in vitro, in vivo, or ex vivo.

In yet another aspect, provided is a method of treating a bacterial infection in a subject in need thereof, the method including administering to the subject any of the disclosed peptides, vectors, or cells. In some aspects, the subject can be mammalian. In some aspects, the subject can be human.

### EXAMPLES

The following examples are put forth so as to provide those of ordinary skill in the art with a complete disclosure and description of how the compounds, compositions, articles, devices, and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Due to its essential role in virulence and high conservation between genera, there is a growing interest to develop therapeutics that target T3SS gene expression, T3SS effectors activity, or T3SS apparatus assembly. Several groups have hypothesized that under appropriate conditions, unlike antibiotics, T3SS inhibitors that target the apparatus critical for virulence without directly killing bacteria may exhibit a lower likelihood of evolutionary pressure for resistance. There are currently three main strategies used to target the T3SS: i) discovery of small molecule or natural product inhibitors, ii) vaccine development, and iii) discovery of antibody based drugs. Each of these strategies has its own power and pitfalls. One promising arena for development of T3SS-centered therapeutics that remains poorly explored is de novo protein design, which has experienced a revolution in recent years due to the application of cutting-edge machine-learning based algorithms.

The main solvent accessible feature of the T3SS apparatus is the needle that forms a channel by which virulence factors are injected into the host cell. The T3SS needle is formed by ~120 copies of a single needle protein that polymerizes through conserved amino acids in the C-terminal α₂-helix. Here, a study was conducted which developed a pipeline to generate “new to nature” de novo protein design inhibitors of T3SS assembly by targeting polymerization hotspots of the Salmonella T3SS needle protein, PrgI, as a model system. The study applied physics-based software RosettaDesign and machine-learning based-software RFDiffusion to design α-helical bundle backbone structures tailored to interact PrgI at surfaces critical for T3SS needle polymerization, and ProteinMPNN to design idealized protein sequence that fold into the desired structure. Twenty-four “rdmpn” and “rosempn” designs successfully expressed in E. coli and folded into stable α-helical bundles. Binding affinities and binding modes for designs with PrgI were experimentally validated. Several designs exhibited the ability to inhibit polymerization of PrgI T3SS needle in vitro. The study further showed these designed proteins can associate with homologous needle proteins, Burkholderia BsaL and Shigella MxiH, through binding to conserved surfaces, suggesting the potential for universal T3SS needle targeting. Together, these results provide a molecular blueprint to develop T3SS needle binding proteins with atomic level accuracy, enabling new therapeutic strategies with potential to be applied across T3SS harboring bacteria.

### Materials and Methods

Approach I: For approach I, the study targeted a major PrgI surface involved in intermolecular interaction between adjacent PrgI-PrgI subunits within the Salmonella T3SS needle using a cryo-EM structure of the needle-tip complex (PDB ID 7RYE) This PrgI-PrgI interaction is mediated by residues 58-80 (α₂ helix) on the first PrgI subunit and residues 40-66 (α₂ helix) on the adjacent PrgI subunit. The study first exacted two PrgI subunits (chain D and chain N from PDB ID 7RYE). Chain D was kept fixed to serve as the “target”, and the De novo design of “rdmpn” and “rosempn” proteins: α₂ helix (residues 40-66) from chain N was used as the “motif” to generate α-helical bundles that would bind to the target PrgI. RosettaDesign was carried out following methods described by Cao et al., Briefly, the study defined a variety of three α-helix bundle using the RosettaRemodel blueprint format with the PrgI α₂ helix motif incorporated into one helix of the bundle. Blueprints that differ in lengths of the helices and loop types were generated. Blueprints were then used to generate backbone structures using the Rosetta Monte Carlo-based fragment assembly protocol, while keeping the motif fixed. Before sequence design, the PrgI structure was loaded into a hashing grid for fast clash checking to guarantee the newly generated backbones are not clashing with the target PrgI. Amino acid sequences for each α-helix bundle mini-protein design were prepared with ProteinMPNN using the Colab implementation. For ProteinMPNN, PrgI chain D was kept fixed (fix_pos: D, rm_aa: C) while the designs underwent sequence design with parameters model_name=v_48_020, num_seqs=64 sampling_temp=0.1. For each sequence design, AlphaFold2 was used to validate whether the ProteinMPNN designed sequence was predicted to fold into the desired α-helix bundle. Designs created by this approach are termed “rosempn”. For example, the design rosempn.2_1 is a RosettaDesign where 2 is a random number given for a unique backbone design and 1 is a random number given to a unique ProteinMPNN sequence. rosempn.2_2 would have the same backbone but different amino acid sequence.

Approach II: For approach II, chain D from PDB ID 7RYE was extracted and used as a binding target to diffuse backbone structures of binders. RFDiffusion was used to generate designs via the Colab notebook. The notebook contains a pipeline containing RFDiffusion for backbone design, ProteinMPNN for sequence design, and AlphaFold2 for structure prediction of designed proteins. Several rounds of designs were performed with different PrgI hotspots. For round 1 design backbones 1-4, hotspots were chosen to sterically hinder the interaction between PrgI (chain D) and PrgI (chain N). Diffused backbones were generated with parameters contigs=D: 90-150, pdb=7RYE, copies=1, with Advanced Settings parameters iterations=200, symmetry=none, and hotspot=D62,D55,D44,D34,D27. For round 2 design backbones 1-4, hotspots were chosen to sterically hinder the interaction between PrgI-D and PrgI-N. Diffused backbones were generated with parameters contigs=D3-80:90-150, pdb=7RYE, iterations=50, hotspot=D62,D55,D44,D34,D27, num_designs=1, visual=interactive, symmetry=cyclic, order=1, chains=(blank), and add potential=true. For round 2 backbones 5-9, hotspots were chosen to sterically hinder the interaction between PrgI (chain D) and PrgI (chain N). Diffused backbones were generated with parameters contigs=D3-80:90-150, iterations=100, hotspot=D32,D47,D62,D60, and symmetry=auto. For ProteinMPNN, PrgI chain D was kept fixed (fix_pos: D, rm_aa: C) while the designs underwent sequence design with parameters model_name=v_48_020, num_seqs=64 sampling_temp=0.1. Designs created by this approach are termed “rdmpn”. For example, the design rdmpn.r1.4_1 is a RFDiffusion round 1 design where 4 is a random number given for a unique backbone design and 1 is a random number given to a unique ProteinMPNN sequence. rdmpn.r1.4_2 would have the same backbone but different amino acid sequence.

For both design approaches, designs with high pLDDT confidence scores (>85) and low RMSDs (<1.5 Å) that match the intended designs are selected. The study prioritized designed proteins that were helical bundles containing at least three α-helices due to their high thermal stability.

PrgI/design interface analysis: Following design, PrgI/design complex interfaces were analyzed in silico using two complementary approaches explore the potential of interfaces with suboptimal properties. First, the PrgI/design models were used as input for PDBePISA where solvation free energy gained upon formation of the interface (ΔGⁱ in kcal/mol) and interface area (Å²) were extracted. Second, the PrgI/design models were used as input for Rosetta InterfaceAnalyzer where ΔG separated (in Rosetta energy units, REU) and ΔSASA interface area (Å²) were extracted. The 28 “rdmpn” and “rosempn” designs tested in vitro were chosen to represent a diverse range of interface energies and interface areas.

Molecular Dynamics (MD) simulations: All-atom MD simulations performed in explicit solvent were carried out in GROMACS v.2021.5 using the CHARMM36 force field and TIP3P water model. Simulations were set up using the CHARMM-GUI solution builder. An integration time step of 2 fs was used with coordinates output every 10 ps. The LINCS algorithm was used to constrain H-bonds. The thermodynamic ensemble was nPT where temperature was kept constant at 298.15 K. Nosé-Hoover coupling method with a tau-t of 1 ps was used to maintain temperature. An isotropic Parrinello-Rahman method with a tau-p of 5 ps and a compressibility of 4.5×10⁻⁵ bar⁻¹ was used for pressure coupling. Short range interactions were treated with a Verlet cutoff scheme with 10 Å electrostatic and van der Waals cutoffs and long-range electrostatics were treated using the Particle Mesh Ewald method. Periodic rectangular boundaries were used. Trajectories for each molecule were acquired for 100 ns. Root mean squared deviation (RMSD) graphs (calculated for backbone atoms) were performed in GROMACS using the gmx rms command and C_{α}-C_{α} distance measurements were performed in VMD (1.9.4a57) using the Labels tools.

Expression and Purification of Design Proteins: Synthetic DNA sequences encoding each designed protein were codon optimized for E. coli K12 strains and synthetic DNA was purchased from GenScript. All constructs carried a C-terminal linker (GSWS), TEV cleavage site (ENLYFQGH), His₆-GB1 tag, and were cloned into pET-22b(+) with NdeI/XhoI restriction sites. The resulting DNA plasmids were transformed into E. coli BL21 (DE3) cells. Transformed cells were plated on LB-agar plates containing 1× carbenicillin and grown overnight at 37° C. Freshly transformed cells were then transferred to 1 L of LB Broth (Miller) containing 1× carbenicillin and expressed for ~18 hours overnight at 37° C. at 225 r.p.m. using the Studier autoinduction method. Briefly, 50 mL of autoinduction buffer 1 [0.5 M (NH₄)₂SO₄, 1 M KH₂PO₄, 1 M NaHPO₄ pH 7], 20 mL of autoinduction buffer 2 [25% v/v glycerol, 2.5% w/v D-glucose, 10% w/v α-lactose], and 1 mL of autoinduction buffer 3 [1 M MgSO₄] were added to 1 L of autoclaved LB media supplemented with 1× carbenicillin. Following overnight growth, bacterial cells were harvested by centrifugation at 6,000 r.p.m. for 20 minutes. Spent cell media was discarded and the resulting cell pellet was resuspended in 50 mL Nickel Wash Buffer (300 mM NaCl, 50 mM Tris, and 10 mM imidazole pH 8). To inhibit proteases, 200 μL of 200 mM phenylmethylsulfonyl fluoride was added to the resuspended cells. Cells were then lysed via sonication on ice at 40% amplification with 30 sec pulse on and 30 sec pulse off for a total of 5 minutes. The sonicated cell suspension was centrifuged at 10,000 r.p.m. for 20 minutes, and the supernatant was transferred into the fresh tube. The supernatant was purified by nickel affinity chromatography (HisTrap FF 5 mL, Cytiva) at a flow rate of 5 mL/min using an ÄKTA Go FPLC system. Following loading of the sample, the column was washed with 5 column volumes of with Buffer A (300 mM NaCl, 50 mM Tris, 10 mM imidazole buffer pH 8). Protein was eluted using a linear gradient from 0 to 100% of Buffer B (300 mM NaCl, 50 mM Tris, 500 mM imidazole pH 8) over 10 column volumes. The fractions containing design proteins were then concentrated to 500 μL with 3 kDa Amicon Ultra Centrifugal Filters. Proteins were further purified by a size exchange chromatography with a Superdex 200 Increase 10/300 GL column at flow rate of 0.5 mL/min in buffer (100 mM NaCl, 20 mM sodium phosphate pH 7.2). Protein purify was confirmed with SDS-PAGE. Protein concentration was measured using a NanoDrop with extinction coefficients calculated with ProtParam.

Expression and Purification of T3SS Needle Proteins: A previously described construct of PrgI, termed PrgI* (V65A, V67A), was used since the mutation slows the rate of in vitro polymerization (enabling biophysical characterization), while still being functional in invasion assays. The strategy was adapted for other T3SS needle proteins XX. The V65A, V67A mutations are not expected to largely affect binding with designed proteins. Briefly, DNA encoding for the full length PrgI* (with V65A, V67A mutations) was codon optimized for E. coli K12 strains and cloned into pET-22b (+) with NdeI/XhoI restriction sites. The PrgI* construct carried an N-terminal His₆-GB1 tag with TEV cleavage site (ENLYFQGH). The synthetic DNA plasmid was transformed into E. coli BL21 (DE3) cells. Freshly transformed cells were plated on LB-agar plates containing 1× carbenicillin and grown overnight at 37° C. To prepare a starter culture, a single bacterial colony was added to 15 mL LB media with 1× carbenicillin and grown overnight at 37° C. at 225 r.p.m . . . . In the morning, the starter culture was added to 1 L of LB-broth with 1× carbenicillin and grown at 37° C. 225 r.p.m. until an OD₆₀₀ of 0.6. Next, 250 mg Isopropyl β-D-1-thiogalactopyranoside (IPTG) was added (final concentration 1 mM) and the culture was grown for 37° C. at 225 r.p.m. for 5 hrs.

Spent cell media was discarded and the resulting cell pellet was resuspended in 50 mL Nickel Wash Buffer (300 mM NaCl, 50 mM Tris, 10 mM imidazole pH 8). To inhibit proteases, 200 μL of 200 mM phenylmethylsulfonyl fluoride was added to the resuspended cells. Cells were then lysed via sonication on ice at 40% amplification with 30 sec pulse on and 30 sec pulse off for a total of 5 minutes. The sonicated cell suspension was centrifuged at 10,000 r.p.m. for 20 minutes, and the supernatant was transfer into the fresh tube. The supernatant was purified by nickel affinity chromatography (HisTrap FF 5 mL, Cytiva) at a flow rate of 5 mL/min using an ÄKTA Go FPLC system. Following loading of the sample, the column was washed with 5 column volumes of with Buffer A (300 mM NaCl, 50 mM Tris, 10 mM imidazole buffer pH 8). Protein was eluted using a linear gradient from 0 to 100% of Buffer B (300 mM NaCl, 50 mM Tris, and 500 mM imidazole buffer pH 8) over 10 column volumes. The fractions containing T3SS needle proteins were then concentrated to 500 μL with 3 kDa Amicon Ultra Centrifugal Filters. The His-GB1-TEV tag was cleaved off with TEV protease (1:100 PrgI*:TEV molar ratio) in TEV buffer (100 mM NaCl, 50 mM Tris pH 8, 0.5 mM EDTA, 1 mM DTT) overnight at 4° C. T3SS needle proteins (now cleaved from the purification tag) were further purified by a second round of nickel affinity chromatography. Purified T3SS needle proteins were extensively dialyzed into 100 mM NaCl, 20 mM sodium phosphate pH 7.2. Protein purify was confirmed with SDS-PAGE. Protein concentration was measured using a NanoDrop with extinction coefficients calculated on ProtParam. Immediately after purification, T3SS needle proteins were flash frozen in liquid nitrogen and stored at −80° C. until use to prevent polymerization.

Circular Dichroism (CD) Spectroscopy: Far-UV CD spectra were collected on a JASCO J-815 CD spectrometer. CD spectra were measured from 350 to 190 nm with 0.3 to 0.5 mg/mL protein in CD buffer (100 mM NaCl, 20 mM sodium phosphate pH 7.2) with 1 mm path length quartz cuvettes (Starna Cells #21-Q-1/CD). CD spectra were acquired in triplicate at 25° C. with a scan rate of 50 nm/min and then averaged. The average CD spectra were smoothed with a Savitzky-Golay filter with convolution width of 7. CD thermal denaturation measurements were acquired (one replicate) with CD signal at 222 nm measured from 25 to 95° C. with an increase of 1° C. min-1. Thermal melt values were determined by fitting with Boltzmann Sigmoidal equations in GraphPad Prism (v10).

Surface Plasmon Resonance: Towards biotinylation of PrgI, a BirA biotin ligase recognition site (GSLHHILDAQKMVWNHR) was appended to the C-terminus of the PrgI* construct. PrgI*-BirA was biotinylated using BirA500 biotin-protein ligase standard reaction kit (Avidity), where the regents were added according to the product's protocol. SPR experiments were conducted in triplicate (n=3) using a BiaCore T200 instrument (Cytiva) in SPR buffer (100 mM NaCl, 20 mM sodium phosphate pH 7.2, 0.05% Tween-20). Approximately 200-350 resonance units (RU) of biotinylated-PrgI* was immobilized on a streptavidin-coated chip (GE Healthcare). Design proteins were flowed over the PrgI* coated surface followed by a wash-out step with buffer. The following protein concentrations were used: 0, 10, 50, 100 and 200 μM. In all experiments samples were injected over the chip at 25° C. at a flow rate of 50 μL min-1 for 60 sec followed by a buffer wash with 180 sec dissociation time and equilibrium data were collected. SPR sensorgrams and equilibrium dissociation constants K_{D} values were analyzed in BiaCore T200 evaluation software 3.1 (Cytiva) using surface-bound steady state kinetic affinity analysis assuming a 1:1 stoichiometry. SPR sensorgrams were prepared in GraphPad Prism v10.

Solution NMR Spectroscopy: To generate uniformly ¹⁵N-labeled PrgI*, MxiH*, and BsaL*, the above expression and purification procedures were followed except that standard 1×M9 minimal media supplemented with ¹⁵N ammonium chloride (Cambridge Isotope Labs) and natural abundance (¹²C) D-glucose was used. Following growth, bacterial cells were harvested by centrifugation at 6,000 r.p.m. for 20 minutes. PrgI* and designed proteins were exhaustively dialyzed into matched NMR buffer (100 mM NaCl, 20 sodium phosphate pH 7.2, 5% D₂O). Solution NMR assignments for PrgI*, MxiH*, and BsaL were transferred from previous reports. Designed proteins (prepared at natural isotopic abundance as described above) were titrated into 100 μM ¹⁵N-labeled PrgI* in a volume of 300 μL. Concentrations used for designs. 2D ¹H-¹⁵N amide HSQC spectra (Bruker pulse sequence XX) were recorded at a ¹H field of 600 MHz at 25° C. with a recycle delay (d1) of 1.2 sec, acquisition time of 0.X see, sweep width of XX ppm, center position of XX ppm, and XX scans on a Bruker AvanceIIIHD-700 spectrometer equipped with 5 mm TCI cryoprobe. Data were processed in NMRPipe and analyzed in NMRFAM-SPARKY. For comparison of free and design-bound PrgI* states, weighted average chemical shift perturbations (CSP, measured in ppm) were determined using the equation Δδ_{NH}=(½ (Δδ_{H}²+Δδ_{N}²/25))^{1/2}. CSP plots were prepared in GraphPad Prism v10.

Transmission Electron Microscopy: Purified PrgI* was dialyzed into 100 mM NaCl, 20 sodium phosphate pH 7.2 for TEM. Following previously established protocols, 50 μL of PrgI* at 0.8 mg/mL was polymerized in vitro at 25° C. for 1 week without shaking. The PrgI* samples were applied to carbon-coated 400-mesh copper transmission electron microscope (TEM) grids (SPI 2040C) and then gently side-blotted using the torn edge of Whatman #4 filter paper. After a single wash with Milli-Q ultrapure water followed by blotting, the grids were negatively stained with 1% uranyl acetate. The grids were blotted again after a 30-second incubation period. The samples were screened using a JEOL JEM-1400 transmission electron microscope at an accelerating voltage of 120 kV. Images were collected at a nominal magnification of 50,000× with a Gatan OneView camera.

### Results

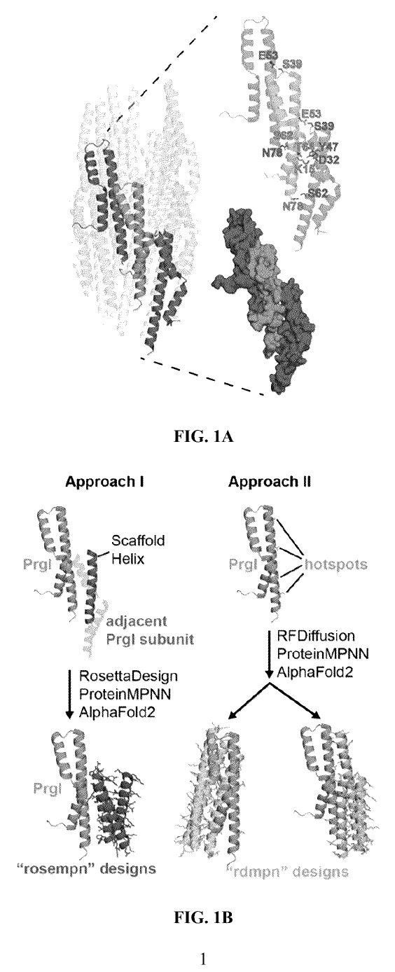

In silico design and characterization of de novo designed PrgI binders: The study set out to generate high affinity protein binders to the Salmonella T3SS needle protein PrgI with the goal to sterically block binding of adjacent PrgI units, which would in theory prohibit polymerization into the T3SS needle. The study explored two approaches to design stable α-helical bundle mini-proteins based on rational, structure-guided design enabled by the cryo-EM structure of the assembled Salmonella T3SS needle (FIGS. 1A-1B). In the first approach (“rosempn”), the α-helix of an adjacent PrgI subunit is used as a “scaffold” that was incorporated into the backbone structure of designed coiled coil. Here, RosettaDesign is used to generate backbone structures and ProteinMPNN is used to design amino acid sequences. Design sequences are optimized both at the interaction interface with PrgI and throughout the entire design. In the second approach (“rdmpn”), hotspots along PrgI α1 or α2 helices that are critical for T3SS needle polymerization are targets for binder design where backbones structures are generated by a full denoising diffusion process via RFDiffusion and the corresponding amino acid sequences are produced by ProteinMPNN. Through the use of diffusion models rather than scaffolded design, the second approach enables sampling of a wider conformational space, both in the type of designs and the molecular surfaces targeted. Following the initial design stage of both approach I and approach II, AlphaFold2 is used towards in silico validation that designed sequences folded into the intended backbone structures.

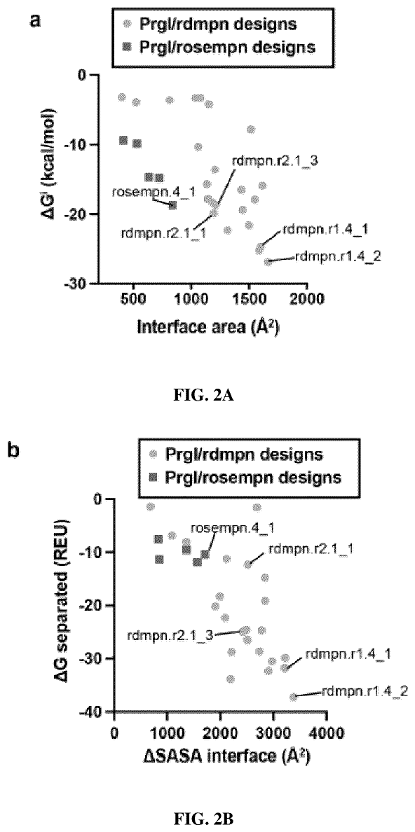

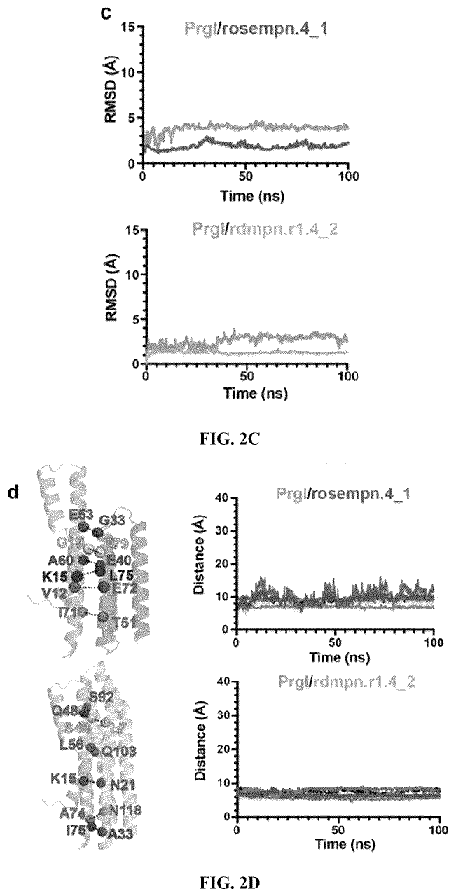

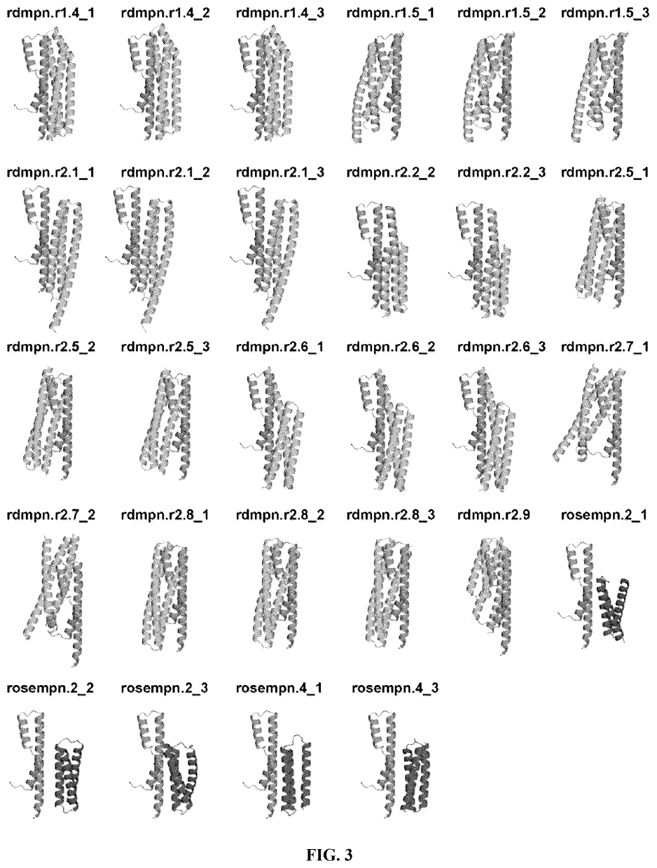

As an additional quality control filter for this pipeline, the study sought to further probe PrgI/design complexes in silico prior to experimental characterization. The study examined the interface energies and interface areas of each PrgI/design complex using two complementary approaches, PDBePISA and Rosetta InterfaceAnalyzer. Overall, the PrgI/design models sampled a diverse landscape of interfaces energies and interface areas (with “rdmpn” designs encompassing a wide structural space) that mimic those observed for biological protein/protein complexes found in nature (FIGS. 2A-2B). To further evaluate the in silico stability of the PrgI/design models, the study performed all-atom molecular dynamics simulations in explicit solvent with trajectories of 100 nsec. Backbone root mean square deviation (RMSD) plots and C_{α}-C_{α} distance measurements between residues spanning the PrgI/design interface provide further evidence of complex stability (FIGS. 2C-2D). Ultimately, a total of twenty-eight “rdmpn” and “rosempn” designs spanning a range of binding modes were chosen for experimental characterization (FIG. 3).

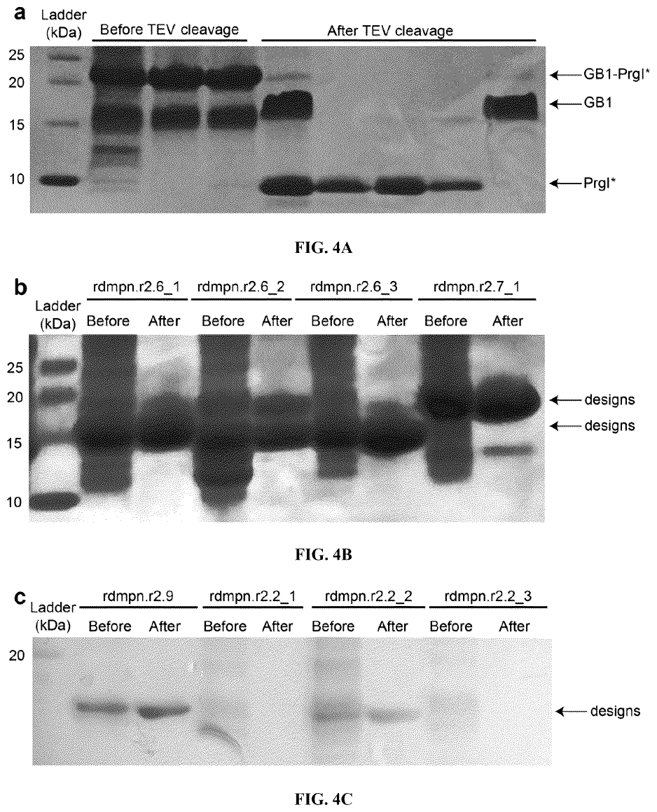

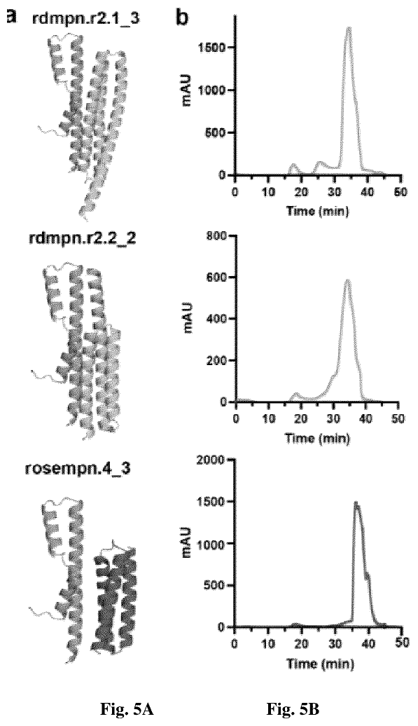

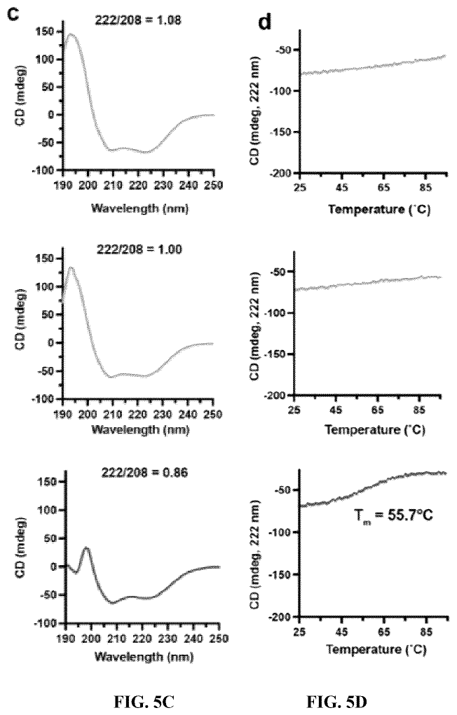

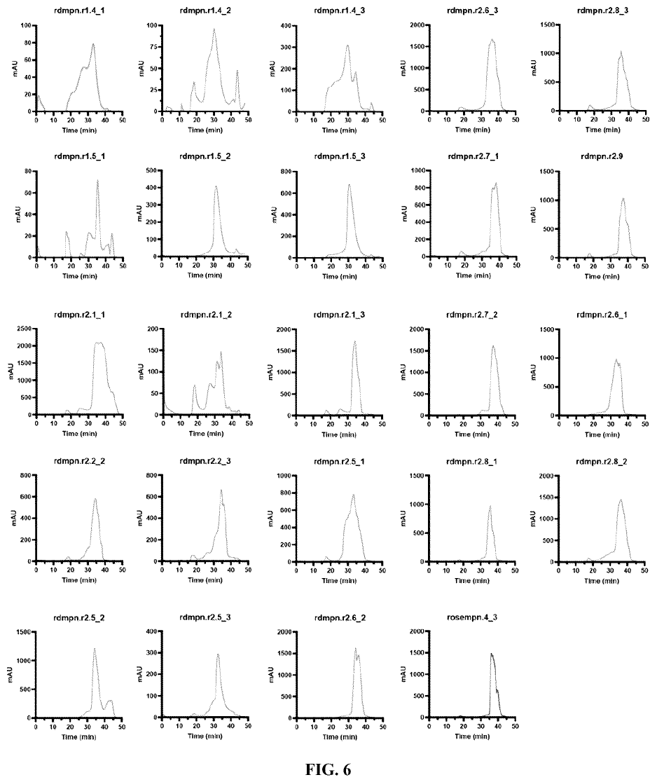

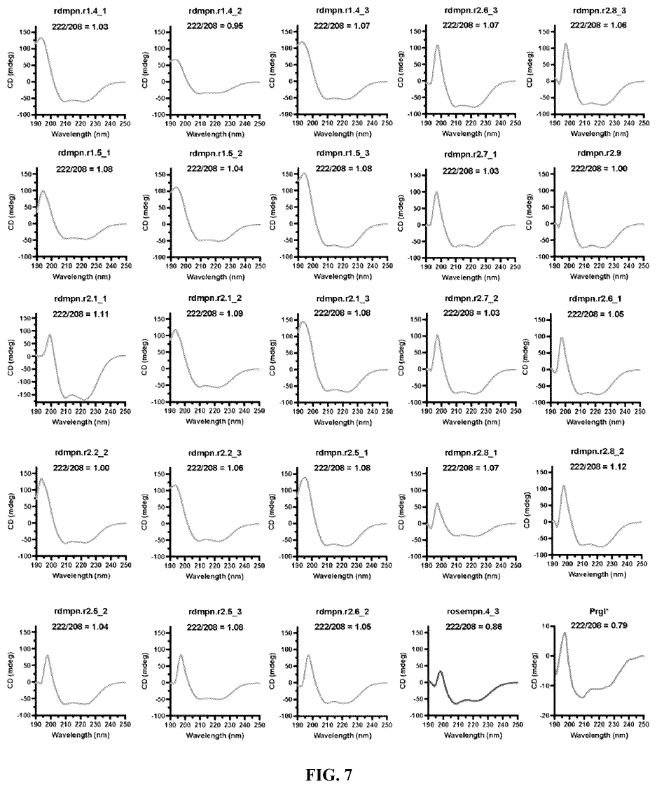

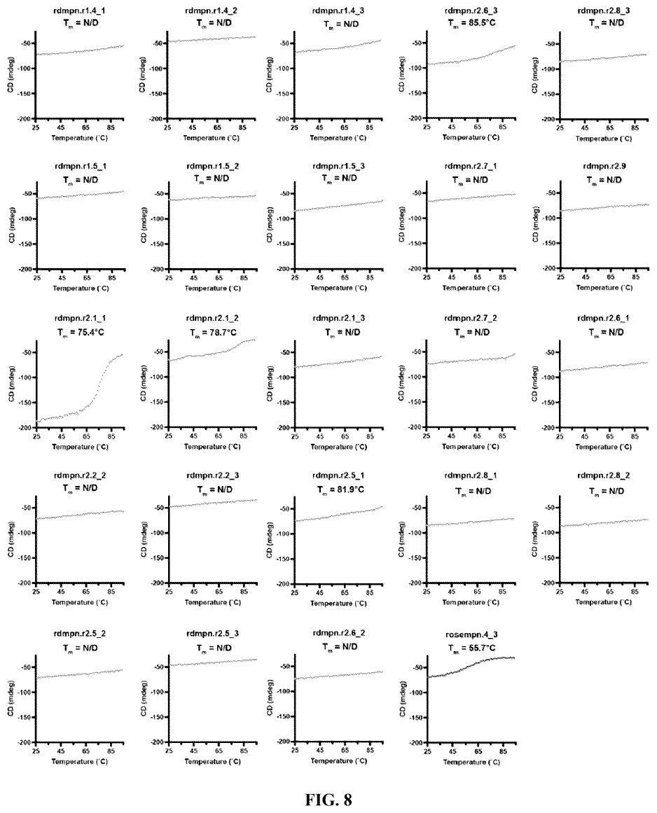

Experimental validation of de novo designed protein expression, refolding, and thermal stability: A soluble, functional version of the Salmonella T3SS needle protein, PrgI*, was recombinantly expressed and purified as previously reported (FIG. 4A). In addition, the twenty-eight de novo designed “rdmpn” and “rosempn” proteins, which serve as candidate binders for monomeric PrgI*, were recombinantly expressed in E. coli. Twenty-four designs were successfully expressed and purified as soluble proteins (FIG. 4B). Size exclusion chromatography (SEC) revealed “rdmpn” and “rosempn” proteins to be well-behaving, soluble species (FIGS. 5A-5B and FIG. 6). Far-UV circular dichroism (CD) spectroscopy showed that designed proteins folded into α-helical secondary structures, as highlighted by the two negative bands at 208 and 222 nm (FIG. 5C and FIG. 7). The CD derived θ₂₂₂/θ₂₀₈ ratio (>1 for most designs) suggests that the de novo designed proteins are properly folded α-helical coiled coil bundles as intended. CD derived thermal melts also provide evidence that most of the de novo designed proteins exhibit melting temperatures (Tₘ) well above 70° C. (FIG. 5D and FIG. 8), consistent with enhanced thermal stability for computationally designed proteins. The experimental Tₘ of PrgI was determined previously to be ~37° C. Together, these results show that de novo designed rdmpn” and “rosempn” proteins can be expressed as monomeric, properly folded, and thermally stable species.

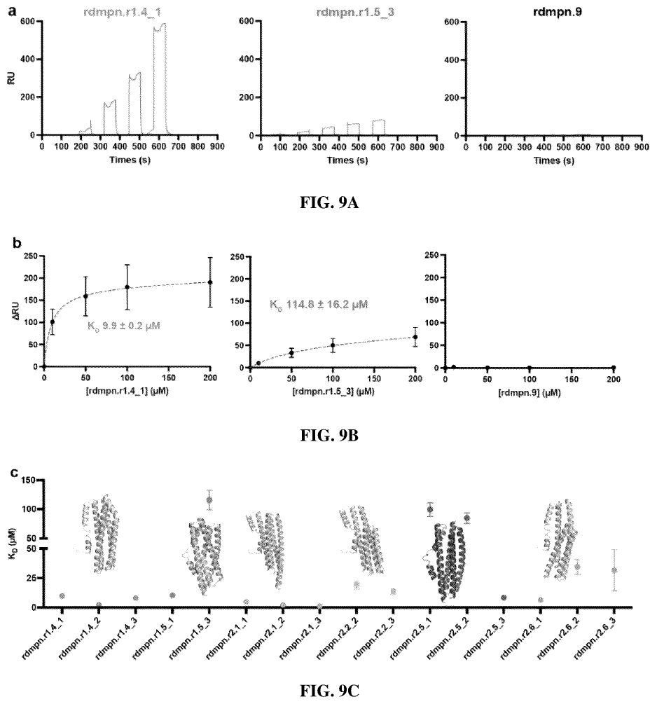

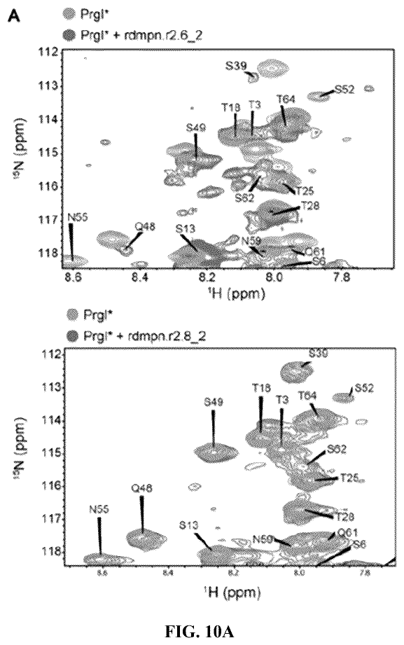

De novo designed proteins bind to monomeric PrgI* with varying affinities: To evaluate whether de novo designed “rdmpn” and “rosempn” proteins could bind to monomeric PrgI*, surface plasmon resonance (SPR) was performed. By flowing increasing concentrations of candidate binder proteins over a streptavidin coated SPR chip immobilized with PrgI*-biotin, dissociation constants (K_{D}) were quantitatively determined. Among the twenty-four de novo designed “rdmpn” and “rosempn” proteins tested, eight showed no detectable binding to PrgI* (FIG. 9 and TABLE 1). For three “rdmpn” proteins, rdmpn.r1.5_3, rdmpn.r2.5_1, and rdmpn.r2.5_2, binding to PrgI* was observed with weak affinity (K_{D}>50 μM) (FIG. 9 and TABLE 1). For four “rdmpn” proteins, rdmpn.r2.2_2, rdmpn.r2.2_3, rdmpn.r2.6_2, and rdmpn.r2.6_3, binding to PrgI* was observed with moderate affinity (K_{D}~15 to 50 μM) (FIG. 9 and TABLE 1). For nine “rdmpn” proteins binding to PrgI* was observed with high affinity (K_{D}<15 μM), with rdmpn.r1.4_1, 4_2, 4_3, rdmpn.r2.1_2, and rdmpn.r2.1_3, as the top designs (FIG. 9 and TABLE 1). Together, these results show that de novo designed proteins bind to monomeric PrgI* with a hit rate of ~66% with KD values ranging from 1.3 to 114.8 μM, suggesting this design pipeline is robust at producing positive binders to T3SS needle proteins.

TABLE 1
SPR derived dissociation constants
(K_{D}) for “rdmpn” and “rosempn”
designs with PrgI*

| Protein binder | K_{D} (μM) |
|---|---|
| rdmpn.r1.4_1 | 9.95 ± 0.15 |
| rdmpn.r1.4_2 | 2.41 ± 0.47 |
| rdmpn.r1.4_3 | 8.07 ± 2.11 |
| rdmpn.r1.5_1 | 10.43 ± 1.17 |
| rdmpn.r2.1_1 | 114.85 ± 16.20 |
| rdmpn.r2.1_2 | 4.97 ± 1.99 |
| rdmpn.r2.1_3 | 2.35 ± 0.48 |
| rdmpn.r2.2_1 | 1.35 ± 0.36 |
| rdmpn.r2.2_2 | 19.55 ± 3.37 |
| rdmpn.r2.2_3 | 13.99 ± 2.61 |
| rdmpn.r2.5_1 | 99.18 ± 11.65 |
| rdmpn.r2.5_2 | 85.05 ± 9.54 |
| rdmpn.r2.5_3 | 8.43 ± 1.31 |
| rdmpn.r2.6_1 | 6.55 ± 0.65 |
| rdmpn.r2.6_2 | 34.61 ± 6.40 |
| rdmpn.r2.6_3 | 31.71 ± 17.56 |

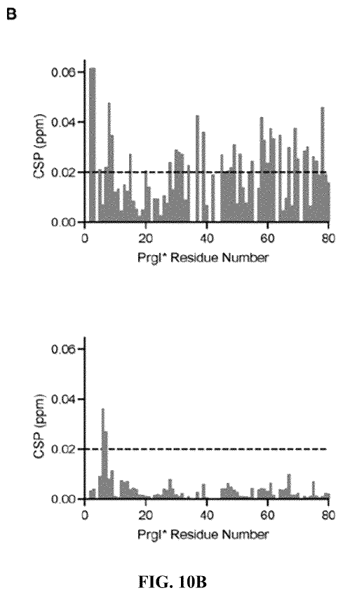

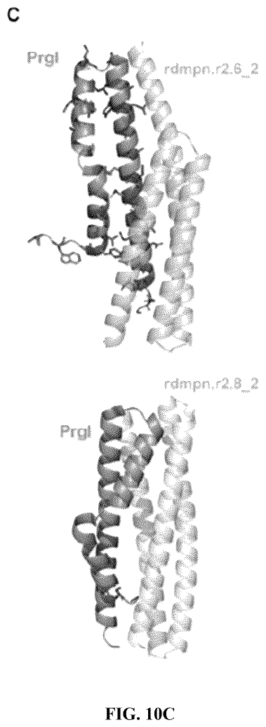

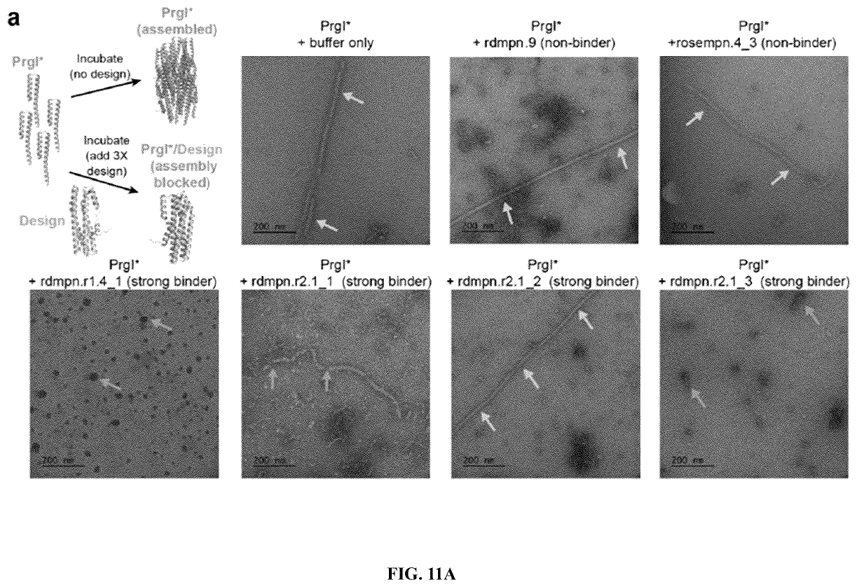

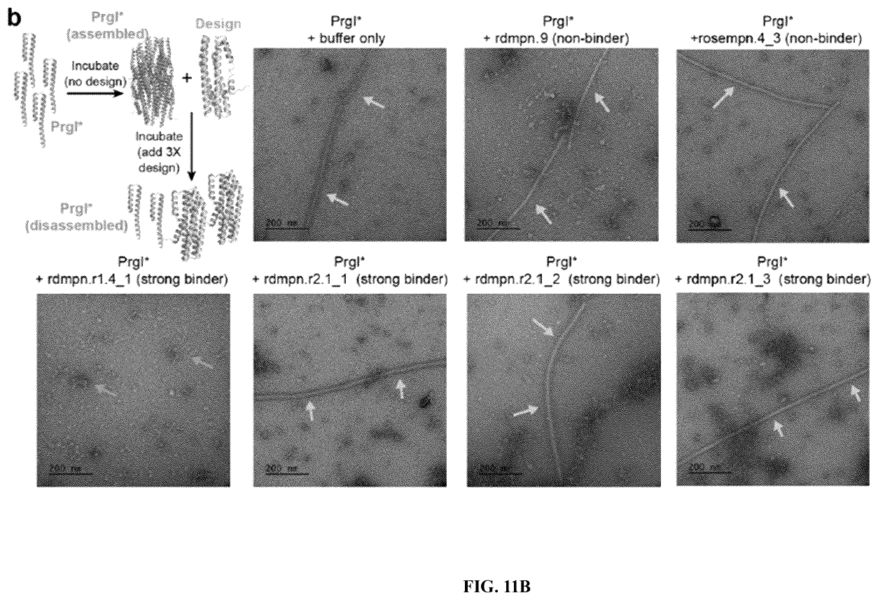

De novo designed proteins bind to monomeric PrgI at the intended molecular surfaces: Upon confirming to binding to PrgI*, the study next asked whether designs bound at the intended molecular surfaces. The study prepared uniformly ¹⁵N-labeled PrgI* with previously established protocols and performed solution nuclear magnetic resonance (NMR) titrations with increasing concentrations of natural isotopic abundance design proteins. When overlaid the HSQC spectra of PrgI in the absence of design proteins, the HSQC spectra of PrgI with designs (2× molar ratio) show shifts for certain amino acids (FIG. 10A). According to the quantitative data of signal shifts, chemical shift perturbations (CSP) were derived and plotted onto PrgI* structure (FIGS. 10B-10C). Comparing the spectra of the two different binders, the relatively stronger binder, rdmpn.r2.6_2, clearly affected and interacted with more amino acids on PrgI, while the weaker binder, rdmpn.r2.8_2, affected only 2 amino acids on PrgI. From those amino acids on PrgI, the amino acids fit to the positions where PrgI subunits bind to other subunits to complete the polymerization process, which confirms that design binders bind to positions on PrgI that serve the purpose of inhibiting polymerization. Among the eight binders, rdmpn. r1.4_1, 5_1, and rdmpn.r2. 1_1, 5_3, 6_1 are strong binders, rdmpn.r1.5_3, and rdmpn.r2. 2_2 are medium binders, and rosempn.4_3 is a nonbinder. To further understand the binding complexes, the study also titrated PrgI proteins with binders and nuclear magnetic resonance (NMR) experiments were conducted.

De novo designed proteins inhibit in vitro PrgI needle polymerization: To explore whether the de novo designed “rdmpn” proteins displayed the intended activity (disruption of T3SS needle assembly), the study turned transmission electron microscopy (TEM) of in vitro polymerized PrgI* needles, which exhibit similar morphology to in vivo T3SS needles. The study first asked whether incubation of 3-fold molar excess of de novo designed proteins with monomeric PrgI* could inhibit the in vitro polymerization of PrgI* into needles (FIG. 11A). PrgI* polymerized into well-defined needles in the absence of any de novo designed proteins, in the presence of non-binding designs, or in the presence of moderate affinity designs (FIG. 11A). In several cases, high affinity binders (i.e., rdmpn.r2.1_1, 1_2, and 1_3) were not able to disrupt in vitro polymerization of PrgI* where well-defined needles could still be observed (FIG. 11A). However, in other cases, high affinity binders were able to either completely block (i.e., rdmpn.r1.4_1) or break/alter the morphology (i.e., rdmpn.r2.1_1) of in vitro polymerization of PrgI* needles (FIG. 11A). The study next asked whether incubation of 3-fold excess of de novo designed proteins with pre-assembled PrgI* needles could result in their disassembly (FIG. 11B). Non-binding designs, moderate affinity binders, and several high affinity binders did not show any ability to disassembly pre-formed PrgI* needles (FIG. 11B). Intriguingly, rdmpn.r1.4_1 exhibited the unique ability to disassemble pre-formed PrgI* needle (FIG. 11B). Together, these results provide evidence that de novo designed “rdmpn” proteins display the ability to inhibit and/or disassemble in vitro polymerized PrgI* needles.

De novo designed proteins bind to homologous T3SS needle proteins through conserved surfaces: T3SS system is a highly conserved effector protein transportation system in Gram-T3SS system is a highly conserved effector protein transportation system in Gram-negative bacterium. By looking into the sequence and structure of needle subunits in different bacterial species, the α₂ alpha helix near the C-terminus is highly conserved. Thus, it was assumed that the PrgI, same with other needle subunits, polymerize into needle structure by binding to the α₂ region of each other. This is also the rationale behind these design approaches.

### DISCUSSION

This work generates de novo designed proteins that bind to monomeric T3SS needle proteins to ultimately block polymerization of the needle. This study presents and experimentally validates a robust pipeline to produce high affinity binders using the Salmonella T3SS needle PrgI as a model system (FIGS. 1A-1B, hit rate ~66%). While most “rosempn” designs (approach I, scaffolded design) exhibited limited solubility and lacked ability to associate with PrgI*, “rdmpn” designs (approach II, hotspot diffusion) expressed in high yields as well-folded thermal stable proteins with diverse structural backbones that exhibited a range of affinities to PrgI* (FIG. 3 and FIG. 9). Most importantly, several designs (i.e., rdmpn.r1.4_1 and rdmpn.r2.1_1) were identified that displayed the ability to inhibit PrgI* needle assembly and/or disassembly pre-formed PrgI* needles in vitro (FIGS. 11A-11B). This study attributes the increased experimental success rates of approach II (“rdmpn” designs) to enhanced sampling of backbone conformational space by diffusion starting from random noise, as suggested previously for RFDiffusion relative RosettaDesign. It is expected that these designs can perform well against wild-type needle proteins. The activity of “rdmpn” designs in in situ models of bacterial infection (i.e., epithelial cell invasion assays) and in vivo mouse models of bacterial pathogen challenge can also be probed.

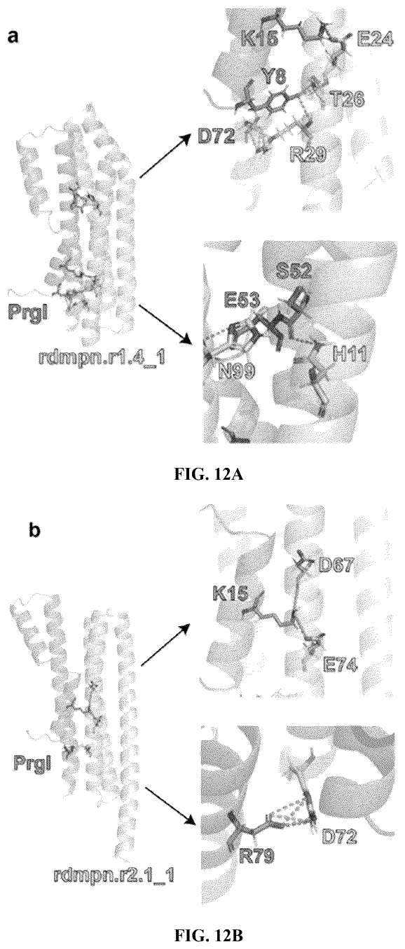

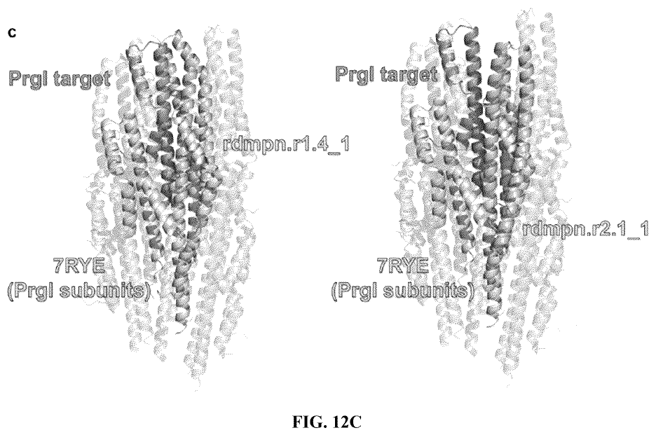

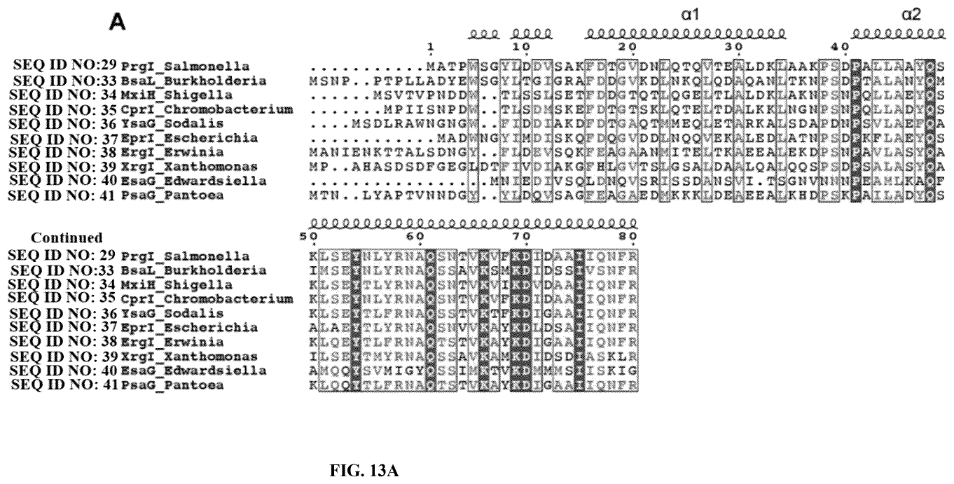

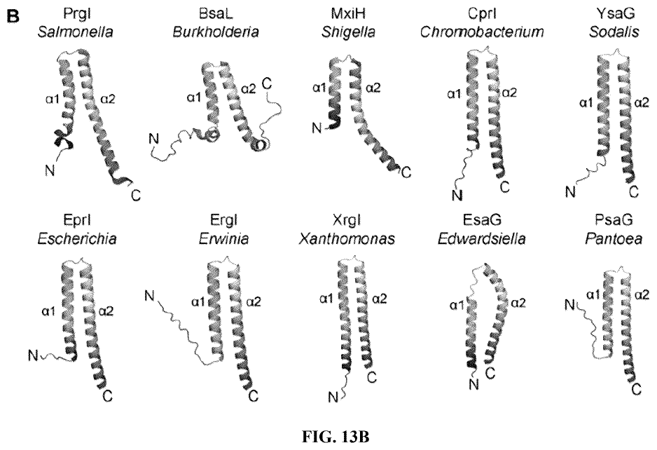

These results help provide initial insights into what distinguishes a design's ability to bind to monomeric T3SS needles to block polymerization. rdmpn.r1.4_1 and rdmpn.r2.1_1 likely exhibit high affinity binding while having the desired activity to disrupt in vitro PrgI* polymerization due to a combination of idealized features including optimized interface energy and interface surface area due to extensive hydrophobic interactions (resulting in high affinity binding), and their ability to envelop the PrgI C-terminal α₂-helix required for polymerization (resulting in steric hindrance of needle polymerization) (FIG. 12). Using RFDiffusion, ProteinMPNN, AlphaFold2, and additional in silico screening, the study was able to obtain μM range T3SS needle protein binders from a single round of design. Multiple rounds of design optimization are typically required to produce designs with sub-μM affinities to targets. Future studies could strengthen the binding of “rdmpn” designs through experimental or in silico methods.

These designs have many of the characteristics of promising candidate de novo designed protein therapeutics including ease of recombinant expression in prokaryotic systems, high thermal stability, and they can be easily tuned to abrogate unintended immune responses as shown for many designed proteins. Observations that “rdmpn” designs can associate with T3SS needle proteins from different bacteria (here, Salmonella, Burkholderia, and Shigella) suggests that targeting conserved surfaces critical for needle polymerization (i.e., the C-terminal α₂-helix) could provide unique opportunities for universal antibacterial therapeutics centered on the T3SS. Finally, these designs could be also useful approaches to tackle public health concerns relating to biological warfare and bioterrorism centered on bacterial pathogens.

### Example Aspects

In view of the described compositions, devices, systems, and methods, herein below are described certain more particular described aspects of the inventions. The particularly recited aspects should not, however, be interpreted to have any limiting effect on any different claims containing different or more general teachings described herein or that the “particular” aspects are somehow limited in some way other than the inherent meanings of the language and formulas literally used therein.

Example 1: A peptide comprising at least 80% sequence identity to any one of SEQ ID NOs: 1-28.

Example 2: The peptide of anyone of the examples herein, particularly Example 1, wherein the peptide comprises at least 90% sequence identity to any one of SEQ ID NOs: 1-28.

Example 3: The peptide of any one of the examples herein, particularly Examples 1-2, wherein the peptide comprises any one of SEQ ID NOs: 1-28.

Example 4: A peptide comprising: an N-terminal alpha helix positioned adjacent to the N-terminus of the peptide; a C-terminal alpha helix positioned adjacent at the C-terminus of the peptide; and a middle alpha helix positioned after the N-terminal alpha helix and before the C-terminal alpha helix; wherein the N-terminal alpha helix is from about 20 to about 25 amino acids in length; wherein the C-terminal alpha helix is from about 20 to about 25 amino acids in length; and wherein the middle alpha helix is from about 20 to about 25 amino acids in length.

Example 5: The peptide of any one of the examples herein, particularly Example 4, wherein the N-terminal alpha helix is joined to the middle alpha helix by a first linker, and wherein the middle alpha helix is joined to the C-terminal alpha helix by a second linker.

Example 6: The peptide of any one of the examples herein, particularly Examples 4-5, wherein the peptide is monomeric.

Example 7: The peptide of any one of the examples herein, particularly Examples 4-6, wherein the peptide is a coiled coil.

Example 8: The peptide of any one of the examples herein, particularly Examples 4-7, wherein the peptide consists of three alpha helices.

Example 9: The peptide of any one of the examples herein, particularly Examples 4-8, wherein the peptide has a dissociation constant (K_{D}) with PrgI from about 1 μM to about 200 μM.

Example 10: The peptide of any one of the examples herein, particularly Examples 4-9, wherein the peptide interacts with one or more of V12, K12, G19, Q48, S49, E53, L56, A60, I71, A74, or 175 of PrgI (SEQ ID NO: 29).

Example 11: The peptide of any one of the examples herein, particularly Example 10, wherein the peptide interacts with five or more of V12, K15, G19, Q48, S49, E53, L56, A60, I71, A74, or 175 of PrgI (SEQ ID NO: 29).

Example 12: The peptide of any one of the examples herein, particularly Example 11, wherein the peptide interacts with ten or more of V12, K15, G19, Q48, S49, E53, L56, A60, I71, A74, or 175 of PrgI (SEQ ID NO: 29).

Example 13: The peptide of any one of the examples herein, particularly Examples 10-12, wherein each amino acid in the peptide which interacts with PrgI is independently from about 5 Å to about 12 Å away from PrgI.

Example 14: The peptide of any one of the examples herein, particularly Examples 4-13, wherein the peptide can be expressed by a bacterium or a yeast.

Example 15: The peptide of any one of the examples herein, particularly Example 14, wherein the peptide can be expressed by E. coli.

Example 16: A peptide comprising three alpha helices, wherein the peptide interacts with one or more of V12, K15, G19, Q48, S49, E53, L56, A60, I71, A74, or 175 of PrgI (SEQ ID NO: 29).

Example 17: The peptide of any one of the examples herein, particularly Example 16, wherein the peptide interacts with five or more of V12, K15, G19, Q48, S49, E53, L56, A60, I71, A74, or 175 of PrgI (SEQ ID NO: 29).

Example 18: The peptide of any one of the examples herein, particularly Example 17, wherein the peptide interacts with ten or more of V12, K15, G19, Q48, S49, E53, L56, A60, I71, A74, or 175 of PrgI (SEQ ID NO: 29).

Example 19: The peptide of any one of the examples herein, particularly Examples 16-18, wherein the peptide comprises: an N-terminal alpha helix positioned adjacent to an N-terminus of the peptide: a C-terminal alpha helix positioned adjacent to a C-terminus of the peptide; and a middle alpha helix positioned after the N-terminal alpha helix and before the C-terminal alpha helix.

Example 20: The peptide of any one of the examples herein, particularly Example 19, wherein the N-terminal alpha helix is from about 20 to about 25 amino acids in length.

Example 21: The peptide of any one of the examples herein, particularly Examples 19-20, wherein the C-terminal alpha helix is from about 20 to about 25 amino acids in length.

Example 22: The peptide of any one of the examples herein, particularly Examples 19-21, wherein the middle alpha helix is from about 20 to about 25 amino acids in length.

Example 23: The peptide of any one of the examples herein, particularly Examples 19-22, wherein the N-terminal alpha helix is joined to the middle alpha helix by a first linker, and wherein the alpha helix is joined to the C-terminal alpha helix by a second linker.

Example 24: The peptide of any one of the examples herein, particularly Examples 16-23, wherein the peptide is monomeric.

Example 25: The peptide of any one of the examples herein, particularly Examples 16-24, wherein the peptide is a coiled coil.

Example 26: The peptide of any one of the examples herein, particularly Examples 16-25, wherein the peptide has a dissociation constant (K_{D}) with PrgI of from about 1 μM to about 200 μM.

Example 27: The peptide of any one of the examples herein, particularly Examples 16-26, wherein each amino acid in the peptide which interacts with PrgI is independently from about 5 Å to about 12 Å away from PrgI.

Example 28: The peptide of any one of the examples herein, particularly Examples 16-27, wherein the peptide can be expressed by a bacterium or a yeast.

Example 29: The peptide of any one of the examples herein, particularly Example 28, wherein the peptide can be expressed by E. coli.

Example 30: A vector encoding the peptide of any of the examples herein, particularly Examples 1-29.

Example 31: A cell comprising the vector of any one of the examples herein, particularly Example 30.

Example 32: The cell of any one of the examples herein, particularly Example 31, wherein the cell is a bacterium or a yeast.

Example 33: The cell of any one of the examples herein, particularly Example 32, wherein the cell is E. coli.

Example 34: A method of inhibiting type 3 secretion system (T3SS) in a bacterium, the method comprising administering to the bacterium the peptide of any of the examples herein, particularly, Examples 1-29.

Example 35: The method of any one of the examples herein, particularly Example 34, wherein the method occurs in vitro, in vivo, or ex vivo.

Example 36: A method of treating a bacterial infection in a subject in need thereof, the method comprising administering to the subject the peptide of any one of the examples herein, particularly Examples 1-29.

Example 37: The method of any one of the examples herein, particularly Example 36, wherein the subject is mammalian.

Example 38: The method of any one of the examples herein, particularly Example 37, wherein the subject is human.

The following patents, applications and publications as listed below and throughout this document are hereby incorporated by reference in their entirety herein.

### REFERENCE LIST

1. Cornelis, G. R. The type III secretion injectisome. Nat. Rev. Microbiol. 4, 811-825 (2006).
2. Deng, W. et al. Assembly, structure, function and regulation of type III secretion systems. Nat. Rev. Microbiol. 15, 323-337 (2017).
3. Worrall, L. J., et al., Structural Insights into Type III Secretion Systems of the Bacterial Flagellum and Injectisome. Annu. Rev. Microbiol. 77, 669-698 (2023).
4. Galán, J. E. & Wolf-Watz, H. Protein delivery into eukaryotic cells by type III secretion machines. Nature 444, 567-573 (2006).
5. Coburn, B., et al., Type III Secretion Systems and Disease. Clin. Microbiol. Rev. 20, 535-549 (2007).
6. Troisfontaines, P. & Cornelis, G. R. Type III secretion: more systems than you think. Physiol. Bethesda Md 20, 326-339 (2005).
7. Antimicrobial Resistance Collaborators. Global burden of bacterial antimicrobial resistance in 2019: a systematic analysis. Lancet Lond. Engl. 399, 629-655 (2022).
8. Bulman, Z. P. et al. Research priorities towards precision antibiotic therapy to improve patient care. Lancet Microbe 3, e795-e802 (2022).
9. Nadeem, S. F. et al. Antimicrobial resistance: more than 70 years of war between humans and bacteria. Crit. Rev. Microbiol. 46, 578-599 (2020).
10. McShan, A. C. & De Guzman, R. N. The Bacterial Type III Secretion System as a Target for Developing New Antibiotics. Chem. Biol. Drug Des. 85, 30-42 (2015).
11. Blasey, N., et al. Targeting bacterial pathogenesis by inhibiting virulence-associated Type III and Type IV secretion systems. Front. Cell. Infect. Microbiol. 12, 1065561 (2022).
12. Hotinger, J. A., et al. Molecular Targets and Strategies for Inhibition of the Bacterial Type III Secretion System (T3SS); Inhibitors Directly Binding to T3SS Components. Biomolecules 11, 316 (2021).
13. Allen, R. C., et al. Targeting virulence: can we make evolution-proof drugs? Nat. Rev. Microbiol. 12, 300-308 (2014).
14. Lv, C. et al. Research Progress on Small Molecular Inhibitors of the Type 3 Secretion System. Molecules 27, 8348 (2022).
15. Fasciano, A. C., et al. Promises and Challenges of the Type Three Secretion System-Injectisome as an Anti-Virulence Target. EcoSal Plus 8, 10.1128/ecosalplus.ESP-0032-2018 (2019).
16. Simonis, A. et al. Discovery of highly neutralizing human antibodies targeting Pseudomonas aeruginosa. Cell 186, 5098-5113.e19 (2023).
17. Buchwald, P. Small-molecule protein-protein interaction inhibitors: therapeutic potential in light of molecular size, chemical space, and ligand binding efficiency considerations. IUBMB Life 62, 724-731 (2010).
18. Frost, I. et al. The role of bacterial vaccines in the fight against antimicrobial resistance: an analysis of the preclinical and clinical development pipeline. Lancet Microbe 4, e113-e125 (2023).
19. Chu, A. E., et al. Sparks of function by de novo protein design. Nat. Biotechnol. 42, 203-215 (2024).
20. Rathinavelan, T. et al. NMR model of PrgI-SipD interaction and its implications in the needle-tip assembly of the Salmonella type III secretion system. J. Mol. Biol. 426, 2958-2969 (2014).
21. Guo, E. Z. & Galán, J. E. Cryo-EM structure of the needle filament tip complex of the Salmonella type III secretion injectisome. Proc. Natl. Acad. Sci. U.S.A 118, e2114552118 (2021).
22. Loquet, A. et al. Atomic model of the type III secretion system needle. Nature 486, 276-279 (2012).
23. Marlovits, T. C. et al. Structural Insights into the Assembly of the Type III Secretion Needle Complex. Science 306, 1040-1042 (2004).
24. Galkin, V. E., et al. The Structure of the Salmonella typhimurium Type III Secretion System Needle Shows Divergence from the Flagellar System. J. Mol. Biol. 396, 1392-1397 (2010).
25. Kenjale, R. et al. The needle component of the type III secreton of Shigella regulates the activity of the secretion apparatus. J. Biol. Chem. 280, 42929-42937 (2005).
26. Wang, Y. et al. Differences in the electrostatic surfaces of the type III secretion needle proteins PrgI, BsaL, and MxiH. J. Mol. Biol. 371, 1304-1314 (2007).
27. Liu, Y. & Kuhlman, B. RosettaDesign server for protein design. Nucleic Acids Res. 34, W235-W238 (2006).
28. Watson, J. L. et al. De novo design of protein structure and function with RFdiffusion. Nature 620, 1089-1100 (2023).
29. Dauparas, J. et al. Robust deep learning-based protein sequence design using ProteinMPNN. Science 378, 49-56 (2022).
30. Cao, L. et al. De novo design of picomolar SARS-COV-2 miniprotein inhibitors. Science 370, 426-431 (2020).
31. Cao, L. et al. Design of protein-binding proteins from the target structure alone. Nature 605, 551-560 (2022).
32. Jumper, J. et al. Highly accurate protein structure prediction with AlphaFold. Nature 596, 583-589 (2021).
33. Stranges, P. B. & Kuhlman, B. A comparison of successful and failed protein interface designs highlights the challenges of designing buried hydrogen bonds. Protein Sci. Publ. Protein Soc. 22, 74-82 (2013).
34. Krissinel, E. & Henrick, K. Inference of macromolecular assemblies from crystalline state. J. Mol. Biol. 372, 774-797 (2007).
35. Chen, J., et al. Protein-protein interactions: General trends in the relationship between binding affinity and interfacial buried surface area. Protein Sci. Publ. Protein Soc. 22, 510-515 (2013).
36. Poyraz, O. et al. Protein refolding is required for assembly of the type three secretion needle. Nat. Struct. Mol. Biol. 17, 788-792 (2010).
37. Guo, E. Z. et al. A polymorphic helix of a Salmonella needle protein relays signals defining distinct steps in type III secretion. PLoS Biol. 17, e3000351 (2019).
38. Lau, S. Y., et al. Synthesis of a model protein of defined secondary and quaternary structure. Effect of chain length on the stabilization and formation of two-stranded alpha-helical coiled-coils. J. Biol. Chem. 259, 13253-13261 (1984).
39. Darboe, N., et al. Physical characterization of MxiH and PrgI, the needle component of the type III secretion apparatus from Shigella and Salmonella. Protein Sci. Publ. Protein Soc. 15, 543-552 (2006).
40. Douzi, B. Protein-Protein Interactions: Surface Plasmon Resonance. Methods Mol. Biol. Clifton NJ 1615, 257-275 (2017).
41. Vázquez Torres, S. et al. De novo design of high-affinity binders of bioactive helical peptides. Nature 626, 435-442 (2024).
42. Gong, H. et al. Characterization of the expression of Salmonella Type III secretion system factor PrgI, SipA, SipB, SopE2, SpaO, and SptP in cultures and in mice. BMC Microbiol. 9, 73 (2009).
43. McShan, A. C., et al. NMR identification of the binding surfaces involved in the Salmonella and Shigella Type III secretion tip-translocon protein-protein interactions. Proteins 84, 1097-1107 (2016).
44. Zhang, J. Z. et al. De novo design of Ras isoform selective binders. 2024.08.29.610300 (2024).
45. Lopez-Morales, J. et al. Protein Engineering and High-Throughput Screening by Yeast Surface Display: Survey of Current Methods. Small Sci. 3, 2300095 (2023).
46. Chevalier, A. et al. Massively parallel de novo protein design for targeted therapeutics. Nature 550, 74-79 (2017).
47. King, C. et al. Removing T-cell epitopes with computational protein design. Proc. Natl. Acad. Sci. U.S.A 111, 8577-8582 (2014).
48. Clark, D. P. & Pazdernik, N. J. Biological Warfare: Infectious Disease and Bioterrorism. Biotechnology 687-719 (2016) doi:10.1016/B978-0-12-385015-7.00022-3.
49. Mirdita, M. et al. ColabFold: making protein folding accessible to all. Nat. Methods 19, 679-682 (2022).
50. Mackerell, A. D. et al. All-atom empirical potential for molecular modeling and dynamics studies of proteins. J. Phys. Chem. B 102, 3586-3616 (1998).
51. Pronk, S. et al. GROMACS 4.5: a high-throughput and highly parallel open source molecular simulation toolkit. Bioinforma. Oxf. Engl. 29, 845-854 (2013).
52. Lee, J. et al. CHARMM-GUI Input Generator for NAMD, GROMACS, AMBER, OpenMM, and CHARMM/OpenMM Simulations Using the CHARMM36 Additive Force Field. J. Chem. Theory Comput. 12, 405-413 (2016).
53. Humphrey, W., et al. VMD: visual molecular dynamics. J. Mol. Graph. 14, 33-38, 27-28 (1996).
54. Studier, F. W. Stable expression clones and auto-induction for protein production in E. coli. Methods Mol. Biol. Clifton NJ 1091, 17-32 (2014).

SEQUENCES

SEQ ID NO: 1 rdmpn.r1.4_1
DETVDKLIESHKNILLNAQKNLFEATKERFEVAIEKGVITKEQAEELFAQIKKIVDEAIEK
SFKLLEEKRPEIRALLDKAFTEGPAHLAELSAKIDALNKEIEEILKEMQKKIADLINKTVIQ
NGSWSENLYFQG
SEQ ID NO: 2 rdmpn.r1.4_2
EEVVKQLEESHYNILRNAQENLFEAFEKRSEVAIKLGVITKEEAEKLFKEIKEIVNKAFDE
IKKLLEDKRPELLALLQKAFTEGPEHLAEYSALVDELNKKIQEILDKMQKEISELINNTVI
KNGSWSENLYFQG
SEQ ID NO: 3 rdmpn.r1.4_3
MEVVDQLEESHYNILRNAQENLVKAFEERMKVAIEKGVITEEQAKELYKKIKEILNEAL
EKQKKLIEESRPELQALLDAAFAKGPALLAEYSAKLSKLNEEINEILEEMQKKISELINAT
FIQNGSWSENLYFQG
SEQ ID NO: 4 rdmpn.r1.5_1
SAAALAAAAKAAAEEAAKKILEELEKTTKELEEKLKKAKELWKKGDLKAALALIKEMI
KLIAASAPKITTLTETILEQEATGSDPEFAKKAKETLEKFEKTENELLDFQASLSAQLAAE
VAAGSWSENLYFQG
SEQ ID NO: 5 rdmpn.r1.5_2
SLLEKLKEAKEKAREEAKKILEELKKTTKEIKKLLKEAKKAVEAGDLEKALKLLSEIIKLI
ADSAPKVTKLTETILYLKATGEDPEFAKEAEKTLKDFEAAENDVLSFLSSLTASLLAQVA
AGSWSENLYFQG
SEQ ID NO: 6 rdmpn.r1.5_3
SLEELLKKLEEEREEEAKKNHEELKKTTKEMKELLKKAKELVKKGDLKAALAKLKEIL
KLIAASEPILTSLSEYILYLEATGKDPEFAKEMKETVKEYEEVEKEVLDFLAALTAALLA
QVAAGSWSENLYFQG
SEQ ID NO: 7 rdmpn.r2.1_1
MEELEKLKKEKEWEEEKKKKVEEFAKELAEASKELNDKRNKKIEEIKKTATPAEKKELD
AKSKTFEDAVKIANEALLERVREAMELSKDFSEEVKEQMLKQLKEASKKLLDILTDTSD
SLLALWEKRVKGSWSENLYFQG
SEQ ID NO: 8 rdmpn.r2.1_2
SSLLEELKKEKEQKEEIEKKVKEFEEKLKKKKEELNKKVDKKIDEIKKTASDAFKKELDA
AKKNLDKAFETLEESFLYRVRLGGELSRDFSEEKKEELYKQLEKFSDKLFDIFNNTADSL
MDLYAKKVAGSWSENLYFQG
SEQ ID NO: 9 rdmpn.r2.1_3
MEKKKEEEEEKKWEEETEKIVEEFKKKFEEKAKELKAKLDARLAAIAKKATPAQLKEL
AAKRDNLLNAINTLKEAYLYRVKEAMKLSKDFSEEEKKKILEELEKKAEELFNIAEDTA
NALAALWEKRVAGSWSENLYFQG
SEQ ID NO: 10 rdmpn.r2.2_2
SLAEAAAAEKRYAEALKKASEKLNELFTELKEKVQKMIADLKETGSVTMEDVEKLAEE
VIKKVEKAAKEAGLPEEEQKIFVEITKDLMDRVIAAMKAIMEQGSWSENLYFQG
SEQ ID NO: 11 rdmpn.r2_3
SLLAEAQAAAARAAALNNAAEEFNKLATELKKKVQEMIKDLKETGSITLEEVEKLAKEF
IEKVKKAAEEAGLSEEEKKIFVEIAKDLAKRIIESLKKIMEQGSWSENLYFQG
SEQ ID NO: 12 rdmpn.r2.5_1
AADAKLGAVFKDLKAFMKKADEKLAEFKKKRNELVELLKTGEKMEEAQKLLEELKKE
IEEKMKEIEENSKKFTAEMKAAGAAPALIDNVNAYSKSVINEFEALLDEAETLLAAAELL
GSWSENLYFQG
SEQ ID NO: 13 rdmpn.r2.5_2
AKDAEKGAVIKEFKKFLKEAEEKLKELKEKYNKLRKLLQTGKKQEEAEKLLKEIEEEAK
KIKEEIKKKSKEISAAMKAAGFPQALITNFENLSNSIINEFDALLDQAKTDLEAAKLKGS
WSENLYFQG
SEQ ID NO: 14 rdmpn.r2.5_3
AADAALGAVFKTLKAVLNDAAAAKAELKKMYNELRKLLKTGEKQEEAKKLLEELKKK
IEAKIKELEAKTKEITAAMKAAGAPQALITNVEALFKAIQNEFEALLESAEVDLAAAELK
GSWSENLYFQG
SEQ ID NO: 15 rdmpn.r2.6_1
KELLEKLKEIYDKHRKKIADVLTEYTTKMNAAIKAGDLEAAKKYVEEGYKEISKLLDEL
AKEVSEFTKDPEIAEKMKKTRDEIKKKIKERLEEYLANNIKQAEEEKAAAAALAAKVAA
GSWSENLYFQG
SEQ ID NO: 16 rdmpn.r2.6_2
SALKEELLAIYNKYREKMVEVLTEYTNKANAALKAGDLEKAKAYVEEGLKELKKLLEE
LAKEVAKKTGDPEVAEKAKKAMEEAEKKIEERLKKWLEDNIAQTAQEKAAAAALAAA
VAAGSWSENLYFQG
SEQ ID NO: 17 rdmpn.r2.6_3
AAALETLKKLFNEHRKKIVDILTKYTTEANAAIKAGDVAKAKEAVEKGLKEINKELDKL
EEEVSKLSGDPAFAAKAKATAEKIKETTEKRLKEWLENNLAQKAAEAAAAAALQAAV
AAGSWSENLYFQG
SEQ ID NO: 18 rdmpn.r2.7_1
SELEELEKELKKALEEATKARKELADLEKKYQKEIQADINEDGKYKELVEELKKKQKEI
DELVLKSAELAQQRAAALAAGDLETAKKLLKEQLELEKKLKELAKEAVEILKKIVDGIK
KDKKEKEGSWSENLYFQG
SEQ ID NO: 19 rdmpn.r2.7_2
AEWEALEKEYKAALAAATATRKAAAALEQQNAKLFQQAADADGKYEADLKKAAELG
KKIDELTLKAAALAQERDALLAAGDKEAAKATLKKLLELLKELKELAEERLKTLQAIVD
AIAADKAAAAGSWSENLYFQG
SEQ ID NO: 20 rdmpn.r2.8_1
TVAAIYAAIQSNSKAYNKALIDSVKLYAEDIKRLKELLKELGKEEAAKKAEELLKKVEE
LKKELEEKAKKNNDLLDNAAARLAAGDPAVAADLKAAQAVFKEMEKIEKEAEKLRRE
ARKIAAEAGSWSENLYFQG
SEQ ID NO: 21 rdmpn.r2.8_2
TVEALMAAIQSNSKNLVNAMIEKVKLYLEQIKYLAELAKELGDEETAKKLEELAKKVEE
LLKELEEKGKELNNALDNAIAALKAGDPTVKDLLKEAQKLFKEMAELEKEAEQLAQEA
RELAAKAGSWSENLYFQG
SEQ ID NO: 22 rdmpn.r2.8_3
DVKAIMAAILAESKALNEAMLNTIELYAEEVKYLAELLKELGKEEVAKKAEELLKEIEKI
LKEAKKKGKELNDALKAAIAALEAGDPKVKELLKKARELFKEIEKLVKEAEKKAQEAR
KLAEEAGSWSENLYFQG
SEQ ID NO: 23 rdmpn.r2.9
SEEKEKLLEELEKERQKLIAQLQAVHAQLVAFALEHAPRLSPEAKAKLGEAVVLSSQLLI
KINDGNVEPEELRKLLEEINKLIKEAIALLDDPELKAELEAWRKEFEALVNGIIELIEEEKK
LKEGSWSENLYFQG
SEQ ID NO: 24 rosempn.2_1
AATEAAIKQFKEFAAKFKEIIKELKSGKVNDPRYEAAAIAAGGDAALAGIRAAFFSKDPA
LKAAAKEAAKKGEELEKAAEERAKGSWSENLYFQG
SEQ ID NO: 25 rosempn.2_2
AEDEKIIKKLKEKKEEFKKKIKEFESGSVNDPRTLLAAEASGADAALEGARIAFFSKNEE
VKKAAKEAAKEGKELEKAAQKRASGSWSENLYFQG
SEQ ID NO: 26 rosempn.2_3
EEIKKKIEEFEKFAKEFKEEIKEAKSGKVNDPRTLAAWMAKGADAALKGAIEAFFSKNE
ELKKAAEEAAKAGKELEKAAEKRWKGSWSENLYFQG
SEQ ID NO: 27 rosempn.4_1
LEEEVKKLAEELSKKVKELIEEVKKKNPQAASGRTVLALEAKTGDMVLEATRKYLESK
DLKVGGEIAKKLFEERLEIAEKVAKEGSWSENLYFQG
SEQ ID NO: 28 rosempn.4_2
LEEKAEKLLKETSKKIEELIKEIEKKNPQASSATIVNKLNSTLGDKFLEATREYLASKDLE
EGSKKLEEISKEALKIAEKAAKEGSWSENLYFQG
SEQ ID NO: 29 PrgI chain D
MATPWSGYLDDVSAKFDTGVDNLQTQVTEALDKLAAKPSDPALLAAYQSKLSEYNLY
RNAQSNTVKVFKDIDAAIIQNFR
SEQ ID NO: 30 C-terminal linker
GSWS
SEQ ID NO: 31 TEV cleavage site
ENLYFQGH
SEQ ID NO: 32 biotin ligase recognition site
GSLHHILDAQKMVWNHR
SEQ ID NO: 33 BsaL Burkholderia
MSNPPTPLLADYEWSGYLTGIGRAFDDGVKDLNKQLQDAQANLTKNPSDPTALANYQ
MIMSEYNLYRNAQSSAVKSMKDIDSSIVSNFR
SEQ ID NO: 34 MxiH Shigella
MSVTVPNDDWTLSSLSETFDDGTQTLQGELTLALDKLAKNPSNPQLLAEYQSKLSEYTL
YRNAQSNTVKVIKDVDAAIIQNFR
SEQ ID NO: 35 CprI Chromobacterium
MPIISNPDWTLSDMSKEFDTGTSKLQTELTDALKKLNGNPSNPQLLADYQSKLSEYNLY
RNAQSNTVKVFKDIDAAIIQNFR
SEQ ID NO: 36 YsaG Sodalis
MSDLRAWNGNGWFIDDIAKDFDTGAQTMMEQLETARKALSDAPDNPSVLAEFQAKLS
EYTLFRNAQSSTVKTFKDIGAAIIQNFR
SEQ ID NO: 37 EprI Escherichia
MADWNGYIMDISKQFDQGVDDLNQQVEKALEDLATNPSDPKFLAEYQSALAEYTLYR
NAQSNVVKAYKDLDSAIIQNFR
SEQ ID NO: 38 ErgI Erwinia
MANIENKTTALSDNGYFLDEVSQKFEAGAANMITELTKAEEALEKDPSNPAVLASYQA
KLQEYTLFRNAQTSTVKAYKDIGAAIIQNFR
SEQ ID NO: 39 XrgI Xanthomonas
MPAHASDSDFGEGLDTFIVDIAKGFHLGVTSLGSALDAALQALQQSPSDPSALASYQAIL
SEYTMYRNAQSSAVKAMKDIDSDIASKLR
SEQ ID NO: 40 EsaG Edwardsiella
MNIEDIVSQLDNQVSRISSDANSVITSGNVNNNPEAMLKAQFAMQQYSVMIGYQSSIMK
TVKDMMMSIISKIG
SEQ ID NO: 41 PsaG Pantoea
MTNLYAPTVNNDGYYLDQVSAGFEAGAEDMKKKLDEAEEALKHDPSKPAILADYQSK
LQQYTLFRNAQTSTVKAYKDIGAAIIQNFR

What is claimed is:

1. A peptide comprising at least 80% sequence identity to any one of SEQ ID NOs: 1-28.

2. The peptide of claim 1, wherein the peptide is capable of inhibiting PrgI needle assembly and/or disassembling formed PrgI needle.

3. The peptide of claim 2 comprising:
an N-terminal alpha helix positioned adjacent an N-terminus of the peptide;
a C-terminal alpha helix positioned adjacent a C-terminus of the peptide; and
a middle alpha helix positioned after the N-terminal alpha helix and before the C-terminal alpha helix;
wherein the N-terminal alpha helix is from 20 to 25 amino acids in length;
wherein the C-terminal alpha helix is from 20 to 25 amino acids in length; and
wherein the middle alpha helix is from 20 to 25 amino acids in length.

4. The peptide of claim 3, wherein the N-terminal alpha helix is joined to the middle alpha helix by a first linker, and wherein the middle alpha helix is joined to the C-terminal alpha helix by a second linker.

5. The peptide of claim 4 wherein the linker comprises SEQ ID NO: 30.

6. The peptide of claim 4, wherein the peptide is monomeric.

7. The peptide of claim 6, wherein the peptide is a coiled coil.

8. The peptide of claim 7, wherein the peptide has a dissociation constant (K_{D}) with PrgI of from about 1 μM to about 200 μM.

9. The peptide of claim 8, wherein the peptide interacts with one or more of V12, K15, G19, Q48, S49, E53, L56, A60, I71, A74, or 175 of PrgI chain D (SEQ ID NO: 29).

10. The peptide of claim 9, wherein the peptide is expressed by a bacterium or a yeast.

11. The peptide of claim 10, wherein the peptide is expressed by E. coli.

12. A method of inhibiting type 3 secretion system (T3SS) in a bacterium, the method comprising administering to the bacterium a peptide comprising at least 80% sequence identity to SEQ ID NOs: 1-28.

13. The method of claim 12, wherein the method occurs in vitro, in vivo, or ex vivo.

14. The method of claim 13, wherein the bacterium is a gram-negative bacterium.

15. The method of claim 14, wherein the bacterium is selected from Shigella, Salmonella, Escherichia, Vibrio, Burkholderia, Yersinia, Chlamydia, Chromobacterium, Sodalis, Erwinia, Edwardsiella, Pantoea, Pseudomonas, and Xanthomonas.

16. The method of claim 15, wherein the peptide is administered in 3-fold molar excess of the bacterium.

17. A method of treating a bacterial infection in a subject in need thereof, the method comprising reducing bacterial virulence by administering a peptide comprising at least 80% sequence identity to SEQ ID NOs: 1-28.

18. The method of claim 17, wherein the subject is a mammal, optionally, wherein the mammal is a human.

19. The method of claim 18, wherein the method further comprises an additional treatment.

20. The method of claim 19, wherein the additional treatment is an antimicrobial agent.


SEQUENCE LISTING (41 sequences)

SEQ ID NO: 1 - protein, 135 aa, synthetic construct
  REGION 1..135: note = Description of sequence: polypeptide, name: rdmpn.r1.4_1
DETVDKLIESHKNILLNAQKNLFEATKERFEVAIEKGVITKEQAEELFAQIKKIVDEAIEKSFKLLEEKRPEIRALLDKAFTEGPAHLAELSAKIDALNKEIEEILKEMQKKIADLINKTVIQNGSWSENLYFQG

SEQ ID NO: 2 - protein, 135 aa, synthetic construct
  REGION 1..135: note = Description of sequence: polypeptide, name: rdmpn.r1.4_2
EEVVKQLEESHYNILRNAQENLFEAFEKRSEVAIKLGVITKEEAEKLFKEIKEIVNKAFDEIKKLLEDKRPELLALLQKAFTEGPEHLAEYSALVDELNKKIQEILDKMQKEISELINNTVIKNGSWSENLYFQG

SEQ ID NO: 3 - protein, 135 aa, synthetic construct
  REGION 1..135: note = Description of sequence: polypeptide, name: rdmpn.r1.4_3
MEVVDQLEESHYNILRNAQENLVKAFEERMKVAIEKGVITEEQAKELYKKIKEILNEALEKQKKLIEESRPELQALLDAAFAKGPALLAEYSAKLSKLNEEINEILEEMQKKISELINATFIQNGSWSENLYFQG

SEQ ID NO: 4 - protein, 133 aa, synthetic construct
  REGION 1..133: note = Description of sequence: polypeptide, name: rdmpn.r1.5_1
SAAALAAAAKAAAEEAAKKILEELEKTTKELEEKLKKAKELWKKGDLKAALALIKEMIKLIAASAPKITTLTETILEQEATGSDPEFAKKAKETLEKFEKTENELLDFQASLSAQLAAEVAAGSWSENLYFQG

SEQ ID NO: 5 - protein, 133 aa, synthetic construct
  REGION 1..133: note = Description of sequence: polypeptide, name: rdmpn.r1.5_2
SLLEKLKEAKEKAREEAKKILEELKKTTKEIKKLLKEAKKAVEAGDLEKALKLLSEIIKLIADSAPKVTKLTETILYLKATGEDPEFAKEAEKTLKDFEAAENDVLSFLSSLTASLLAQVAAGSWSENLYFQG

SEQ ID NO: 6 - protein, 133 aa, synthetic construct
  REGION 1..133: note = Description of sequence: polypeptide, name: rdmpn.r1.5_3
SLEELLKKLEEEREEEAKKNHEELKKTTKEMKELLKKAKELVKKGDLKAALAKLKEILKLIAASEPILTSLSEYILYLEATGKDPEFAKEMKETVKEYEEVEKEVLDFLAALTAALLAQVAAGSWSENLYFQG

SEQ ID NO: 7 - protein, 140 aa, synthetic construct
  REGION 1..140: note = Description of sequence: polypeptide, name: rdmpn.r2.1_1
MEELEKLKKEKEWEEEKKKKVEEFAKELAEASKELNDKRNKKIEEIKKTATPAEKKELDAKSKTFEDAVKIANEALLERVREAMELSKDFSEEVKEQMLKQLKEASKKLLDILTDTSDSLLALWEKRVKGSWSENLYFQG

SEQ ID NO: 8 - protein, 140 aa, synthetic construct
  REGION 1..140: note = Description of sequence: polypeptide, name: rdmpn.r2.1_2
SSLLEELKKEKEQKEEIEKKVKEFEEKLKKKKEELNKKVDKKIDEIKKTASDAFKKELDAAKKNLDKAFETLEESFLYRVRLGGELSRDFSEEKKEELYKQLEKFSDKLFDIFNNTADSLMDLYAKKVAGSWSENLYFQG

SEQ ID NO: 9 - protein, 140 aa, synthetic construct
  REGION 1..140: note = Description of sequence: polypeptide, name: rdmpn.r2.1_3
MEKKKEEEEEKKWEEETEKIVEEFKKKFEEKAKELKAKLDARLAAIAKKATPAQLKELAAKRDNLLNAINTLKEAYLYRVKEAMKLSKDFSEEEKKKILEELEKKAEELFNIAEDTANALAALWEKRVAGSWSENLYFQG

SEQ ID NO: 10 - protein, 112 aa, synthetic construct
  REGION 1..112: note = Description of sequence: polypeptide, name: rdmpn.r2.2_2
SLAEAAAAEKRYAEALKKASEKLNELFTELKEKVQKMIADLKETGSVTMEDVEKLAEEVIKKVEKAAKEAGLPEEEQKIFVEITKDLMDRVIAAMKAIMEQGSWSENLYFQG

SEQ ID NO: 11 - protein, 112 aa, synthetic construct
  REGION 1..112: note = Description of sequence: polypeptide, name: rdmpn.r2_3
SLLAEAQAAAARAAALNNAAEEFNKLATELKKKVQEMIKDLKETGSITLEEVEKLAKEFIEKVKKAAEEAGLSEEEKKIFVEIAKDLAKRIIESLKKIMEQGSWSENLYFQG

SEQ ID NO: 12 - protein, 128 aa, synthetic construct
  REGION 1..128: note = Description of sequence: polypeptide, name: rdmpn.r2.5_1
AADAKLGAVFKDLKAFMKKADEKLAEFKKKRNELVELLKTGEKMEEAQKLLEELKKEIEEKMKEIEENSKKFTAEMKAAGAAPALIDNVNAYSKSVINEFEALLDEAETLLAAAELLGSWSENLYFQG

SEQ ID NO: 13 - protein, 128 aa, synthetic construct
  REGION 1..128: note = Description of sequence: polypeptide, name: rdmpn.r2.5_2
AKDAEKGAVIKEFKKFLKEAEEKLKELKEKYNKLRKLLQTGKKQEEAEKLLKEIEEEAKKIKEEIKKKSKEISAAMKAAGFPQALITNFENLSNSIINEFDALLDQAKTDLEAAKLKGSWSENLYFQG

SEQ ID NO: 14 - protein, 128 aa, synthetic construct
  REGION 1..128: note = Description of sequence: polypeptide, name: rdmpn.r2.5_3
AADAALGAVFKTLKAVLNDAAAAKAELKKMYNELRKLLKTGEKQEEAKKLLEELKKKIEAKIKELEAKTKEITAAMKAAGAPQALITNVEALFKAIQNEFEALLESAEVDLAAAELKGSWSENLYFQG

SEQ ID NO: 15 - protein, 129 aa, synthetic construct
  REGION 1..129: note = Description of sequence: polypeptide, name: rdmpn.r2.6_1
KELLEKLKEIYDKHRKKIADVLTEYTTKMNAAIKAGDLEAAKKYVEEGYKEISKLLDELAKEVSEFTKDPEIAEKMKKTRDEIKKKIKERLEEYLANNIKQAEEEKAAAAALAAKVAAGSWSENLYFQG

SEQ ID NO: 16 - protein, 129 aa, synthetic construct
  REGION 1..129: note = Description of sequence: polypeptide, name: rdmpn.r2.6_2
SALKEELLAIYNKYREKMVEVLTEYTNKANAALKAGDLEKAKAYVEEGLKELKKLLEELAKEVAKKTGDPEVAEKAKKAMEEAEKKIEERLKKWLEDNIAQTAQEKAAAAALAAAVAAGSWSENLYFQG

SEQ ID NO: 17 - protein, 129 aa, synthetic construct
  REGION 1..129: note = Description of sequence: polypeptide, name: rdmpn.r2.6_3
AAALETLKKLFNEHRKKIVDILTKYTTEANAAIKAGDVAKAKEAVEKGLKEINKELDKLEEEVSKLSGDPAFAAKAKATAEKIKETTEKRLKEWLENNLAQKAAEAAAAAALQAAVAAGSWSENLYFQG

SEQ ID NO: 18 - protein, 136 aa, synthetic construct
  REGION 1..136: note = Description of sequence: polypeptide, name: rdmpn.r2.7_1
SELEELEKELKKALEEATKARKELADLEKKYQKEIQADINEDGKYKELVEELKKKQKEIDELVLKSAELAQQRAAALAAGDLETAKKLLKEQLELEKKLKELAKEAVEILKKIVDGIKKDKKEKEGSWSENLYFQG

SEQ ID NO: 19 - protein, 136 aa, synthetic construct
  REGION 1..136: note = Description of sequence: polypeptide, name: rdmpn.r2.7_2
AEWEALEKEYKAALAAATATRKAAAALEQQNAKLFQQAADADGKYEADLKKAAELGKKIDELTLKAAALAQERDALLAAGDKEAAKATLKKLLELLKELKELAEERLKTLQAIVDAIAADKAAAAGSWSENLYFQG

SEQ ID NO: 20 - protein, 135 aa, synthetic construct
  REGION 1..135: note = Description of sequence: polypeptide, name: rdmpn.r2.8_1
TVAAIYAAIQSNSKAYNKALIDSVKLYAEDIKRLKELLKELGKEEAAKKAEELLKKVEELKKELEEKAKKNNDLLDNAAARLAAGDPAVAADLKAAQAVFKEMEKIEKEAEKLRREARKIAAEAGSWSENLYFQG

SEQ ID NO: 21 - protein, 135 aa, synthetic construct
  REGION 1..135: note = Description of sequence: polypeptide, name: rdmpn.r2.8_2
TVEALMAAIQSNSKNLVNAMIEKVKLYLEQIKYLAELAKELGDEETAKKLEELAKKVEELLKELEEKGKELNNALDNAIAALKAGDPTVKDLLKEAQKLFKEMAELEKEAEQLAQEARELAAKAGSWSENLYFQG

SEQ ID NO: 22 - protein, 135 aa, synthetic construct
  REGION 1..135: note = Description of sequence: polypeptide, name: rdmpn.r2.8_3
DVKAIMAAILAESKALNEAMLNTIELYAEEVKYLAELLKELGKEEVAKKAEELLKEIEKILKEAKKKGKELNDALKAAIAALEAGDPKVKELLKKARELFKEIEKLVKEAEKKAQEARKLAEEAGSWSENLYFQG

SEQ ID NO: 23 - protein, 136 aa, synthetic construct
  REGION 1..136: note = Description of sequence: polypeptide, name: rdmpn.r2.9
SEEKEKLLEELEKERQKLIAQLQAVHAQLVAFALEHAPRLSPEAKAKLGEAVVLSSQLLIKINDGNVEPEELRKLLEEINKLIKEAIALLDDPELKAELEAWRKEFEALVNGIIELIEEEKKLKEGSWSENLYFQG

SEQ ID NO: 24 - protein, 95 aa, synthetic construct
  REGION 1..95: note = Description of sequence: polypeptide, name: rosempn.2_1
AATEAAIKQFKEFAAKFKEIIKELKSGKVNDPRYEAAAIAAGGDAALAGIRAAFFSKDPALKAAAKEAAKKGEELEKAAEERAKGSWSENLYFQG

SEQ ID NO: 25 - protein, 95 aa, synthetic construct
  REGION 1..95: note = Description of sequence: polypeptide, name: rosempn.2_2
AEDEKIIKKLKEKKEEFKKKIKEFESGSVNDPRTLLAAEASGADAALEGARIAFFSKNEEVKKAAKEAAKEGKELEKAAQKRASGSWSENLYFQG

SEQ ID NO: 26 - protein, 95 aa, synthetic construct
  REGION 1..95: note = Description of sequence: polypeptide, name: rosempn.2_3
EEIKKKIEEFEKFAKEFKEEIKEAKSGKVNDPRTLAAWMAKGADAALKGAIEAFFSKNEELKKAAEEAAKAGKELEKAAEKRWKGSWSENLYFQG

SEQ ID NO: 27 - protein, 95 aa, synthetic construct
  REGION 1..95: note = Description of sequence: polypeptide, name: rosempn.4_1
LEEEVKKLAEELSKKVKELIEEVKKKNPQAASGRTVLALEAKTGDMVLEATRKYLESKDLKVGGEIAKKLFEERLEIAEKVAKEGSWSENLYFQG

SEQ ID NO: 28 - protein, 95 aa, synthetic construct
  REGION 1..95: note = Description of sequence: polypeptide, name: rosempn.4_2
LEEKAEKLLKETSKKIEELIKEIEKKNPQASSATIVNKLNSTLGDKFLEATREYLASKDLEEGSKKLEEISKEALKIAEKAAKEGSWSENLYFQG

SEQ ID NO: 29 - protein, 80 aa, synthetic construct
  REGION 1..80: note = Description of sequence: polypeptide, name: PrgI chain D
MATPWSGYLDDVSAKFDTGVDNLQTQVTEALDKLAAKPSDPALLAAYQSKLSEYNLYRNAQSNTVKVFKDIDAAIIQNFR

SEQ ID NO: 30 - protein, 4 aa, synthetic construct
  REGION 1..4: note = Description of sequence: polypeptide, name: C-terminal linker
GSWS

SEQ ID NO: 31 - protein, 8 aa, synthetic construct
  REGION 1..8: note = Description of sequence: polypeptide, name: TEV cleavage site
ENLYFQGH

SEQ ID NO: 32 - protein, 17 aa, synthetic construct
  REGION 1..17: note = Description of sequence: polypeptide, name: biotin ligase recognition site
GSLHHILDAQKMVWNHR

SEQ ID NO: 33 - protein, 89 aa, synthetic construct
  REGION 1..89: note = Description of sequence: polypeptide, name: BsaL Burkholderia
MSNPPTPLLADYEWSGYLTGIGRAFDDGVKDLNKQLQDAQANLTKNPSDPTALANYQMIMSEYNLYRNAQSSAVKSMKDIDSSIVSNFR

SEQ ID NO: 34 - protein, 83 aa, synthetic construct
  REGION 1..83: note = Description of sequence: polypeptide, name: MxiH Shigella
MSVTVPNDDWTLSSLSETFDDGTQTLQGELTLALDKLAKNPSNPQLLAEYQSKLSEYTLYRNAQSNTVKVIKDVDAAIIQNFR

SEQ ID NO: 35 - protein, 82 aa, synthetic construct
  REGION 1..82: note = Description of sequence: polypeptide, name: CprI Chromobacterium
MPIISNPDWTLSDMSKEFDTGTSKLQTELTDALKKLNGNPSNPQLLADYQSKLSEYNLYRNAQSNTVKVFKDIDAAIIQNFR

SEQ ID NO: 36 - protein, 85 aa, synthetic construct
  REGION 1..85: note = Description of sequence: polypeptide, name: YsaG Sodalis
MSDLRAWNGNGWFIDDIAKDFDTGAQTMMEQLETARKALSDAPDNPSVLAEFQAKLSEYTLFRNAQSSTVKTFKDIGAAIIQNFR

SEQ ID NO: 37 - protein, 79 aa, synthetic construct
  REGION 1..79: note = Description of sequence: polypeptide, name: EprI Escherichia
MADWNGYIMDISKQFDQGVDDLNQQVEKALEDLATNPSDPKFLAEYQSALAEYTLYRNAQSNVVKAYKDLDSAIIQNFR

SEQ ID NO: 38 - protein, 89 aa, synthetic construct
  REGION 1..89: note = Description of sequence: polypeptide, name: ErgI Erwinia
MANIENKTTALSDNGYFLDEVSQKFEAGAANMITELTKAEEALEKDPSNPAVLASYQAKLQEYTLFRNAQTSTVKAYKDIGAAIIQNFR

SEQ ID NO: 39 - protein, 89 aa, synthetic construct
  REGION 1..89: note = Description of sequence: polypeptide, name: XrgI Xanthomonas
MPAHASDSDFGEGLDTFIVDIAKGFHLGVTSLGSALDAALQALQQSPSDPSALASYQAILSEYTMYRNAQSSAVKAMKDIDSDIASKLR

SEQ ID NO: 40 - protein, 73 aa, synthetic construct
  REGION 1..73: note = Description of sequence: polypeptide, name: EsaG Edwardsiella
MNIEDIVSQLDNQVSRISSDANSVITSGNVNNNPEAMLKAQFAMQQYSVMIGYQSSIMKTVKDMMMSIISKIG

SEQ ID NO: 41 - protein, 87 aa, synthetic construct
  REGION 1..87: note = Description of sequence: polypeptide, name: PsaG Pantoea
MTNLYAPTVNNDGYYLDQVSAGFEAGAEDMKKKLDEAEEALKHDPSKPAILADYQSKLQQYTLFRNAQTSTVKAYKDIGAAIIQNFR